SEQUENCE LISTING (10 sequences)

SEQ ID NO: 1 - protein, 5 aa, Homo sapiens
NYWIG

SEQ ID NO: 2 - protein, 17 aa, Homo sapiens
IIDPSNSYTRYSPSFQG

SEQ ID NO: 3 - protein, 8 aa, Homo sapiens
WYYKPFDV

SEQ ID NO: 4 - protein, 14 aa, Homo sapiens
TGSSSNIGSGYDVH

SEQ ID NO: 5 - protein, 7 aa, Homo sapiens
GNSKRPS

SEQ ID NO: 6 - protein, 11 aa, Homo sapiens
ASWTDGLSLVV

SEQ ID NO: 7 - protein, 117 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSS

SEQ ID NO: 8 - protein, 111 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVL

SEQ ID NO: 9 - protein, 447 aa, Homo sapiens
EVQLVQSGAEVKKPGESLKISCKGSGYSFSNYWIGWVRQMPGKGLEWMGIIDPSNSYTRYSPSFQGQVTISADKSISTAYLQWSSLKASDTAMYYCARWYYKPFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 217 aa, Homo sapiens
QSVLTQPPSVSGAPGQRVTISCTGSSSNIGSGYDVHWYQQLPGTAPKLLIYGNSKRPSGVPDRFSGSKSGTSASLAITGLQSEDEADYYCASWTDGLSLVVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2023/0312703 A1
(43) Pub. Date: Oct. 5, 2023

(54) Method of Treating Psoriasis with IL-23 Specific Antibody

(71) Applicant: Janssen Biotech, Inc., Horsham, PA (US)

(72) Inventors: Daphne Chan, Abington, PA (US); Olivia Choi, Houston, TX (US); Long-Long Gao, Paoli, PA (US); Jenny Jeyarajah, Ontario (CA); Shu Li, Dresher, PA (US); Katelyn Rowland, South Bend, IN (US); Vikash Sinha, Newtown, PA (US); Yanli Wang, Trevose, PA (US)

(21) Appl. No.: 18/128,310

(22) Filed: Mar. 30, 2023

Related U.S. Application Data
(60) Provisional application No. 63/325,454, filed on Mar. 30, 2022.

(51) Int. Cl. C07K 16/24; A61K 45/06; A61P 17/06; A61K 9/00

(52) U.S. Cl. CPC C07K 16/244; A61K 45/06; A61P 17/06; A61K 9/0019; A61K 2039/505

(57) ABSTRACT

A method of treating mild to moderate psoriasis in a patient administers an IL-23 specific antibody, e.g., guselkumab, at an initial dose and subsequent doses in order for the patient to respond to the antibody and meet one or more of the clinical endpoints.

### CROSS-REFERENCE TO RELATED APPLICATIONS

This application claims the benefit of U.S. Provisional Application Ser. No. 63/325,454, filed 30 Mar. 2022. The entire contents of the aforementioned application are incorporated herein by reference in their entireties.

### REFERENCE TO SEQUENCE LISTING SUBMITTED ELECTRONICALLY

The sequence listing of the present application is submitted electronically via The United States Patent and Trademark Center Patent Center as an XML formatted sequence listing with a file name “JBI6708USNP1SEQLIST.xml”, creation date of 10 Feb. 2023, and a size of 11 kilobytes (KB). This sequence listing submitted is part of the specification and is herein incorporated by reference in its entirety.

### FIELD OF THE INVENTION

The present invention is directed to methods of treating mild to moderate psoriasis with an antibody that binds human IL23. In particular, it relates to doses and dosing regimens, uses and methods for administration of an anti-IL23 specific antibody and specific pharmaceutical compositions of an antibody.

### BACKGROUND OF THE INVENTION

Guselkumab (Tremfya® of Janssen Biotech, Inc.) is a fully human immunoglobulin G1 lambda (IgG1lambda) monoclonal antibody (mAb) that binds to the p19 subunit of human interleukin (IL)-23 with high specificity and affinity. The binding of guselkumab to the IL-23p19 subunit blocks the binding of extracellular IL-23 to the cell surface IL-23 receptor, thus inhibiting IL-23-specific intracellular signaling and downstream activation and cytokine production. Guselkumab is currently approved for the treatment of adults with moderate to severe plaque psoriasis or moderate to severe psoriatic arthritis (PsA), in the United States, the European Union, Canada, several countries in Latin America, and the Asia-Pacific region. Guselkumab has also been approved for the treatment of generalized pustular psoriasis, erythrodermic psoriasis, and palmoplantar pustulosis in Japan. In addition, guselkumab is being evaluated in both ulcerative colitis and Crohn's disease and in several other immune-mediated dermatologic and rheumatologic diseases globally.

Plaque psoriasis is a chronic, immune-mediated disorder characterized by raised, well-demarcated erythematous plaques with silvery scales that affects 2-3% of the population. Psoriasis is associated with multiple comorbidities including autoimmune disease, neurological disorders, cardiometabolic diseases and inflammatory bowel diseases. Approximately 80% of the psoriasis population has mild to moderate disease.

Traditionally, severity has been classified into 3 categories: mild, moderate, and severe based on body surface area (BSA) and Psoriasis Area and Severity Index (PAST). Patients with “mild” disease have traditionally been treated with topical therapies while patients with “moderate to severe” disease have been candidates for systemic therapy. Severity eligibility criteria for randomized controlled trials of newer psoriasis therapies for moderate to severe psoriasis have generally been set at 10% or more BSA involvement. US and international dermatology medical societies have assessed that treatment decisions using these artificially designed cut-offs based on quantitative measures such as BSA exclude a majority of patients who are significantly impacted by undertreated disease. For example, it is well established that patients with regional subtypes such as involvement of face, genitalia, palms and soles, scalp and nails can suffer greatly from their localized psoriasis but often do not fit the traditional definition of severe disease if skin involvement is not at least 10% BSA. Both the International Psoriasis Council (IPC) and American Academy of Dermatology (AAD)-National Psoriasis Foundation (NPF) have recently released publications that reject the mild, moderate, and severe categories in favor of a dichotomous definition: candidates for topical therapy or candidates for systemic therapy including biologics.

The IPC proposes that candidates for systemic therapy include those with (1) BSA≥10%, or (2) disease involving special areas (hands/feet, nails, face, scalp, and genitals), or (3) failure of topical therapy. The AAD-NPF guidelines similarly acknowledge that psoriasis can be severe irrespective of BSA when it has serious emotional consequences or when it occurs in select locations, including but not restricted to, the hands, feet, scalp, face, or genital area, or when it causes intractable pruritus.

Consistent with the gap identified above, an NPF survey from 2003-2011 revealed that 30% of patients with moderate psoriasis (defined as 3-10% BSA) received treatment solely with topical medications and 24-36% of these patients felt that they were undertreated. Another study looking at correlations of the Dermatology Life Quality Index (DLQI) with BSA and PASI highlighted the concern that treatment decisions based on threshold assessments of disease measures alone (BSA or PASI) may result in the undertreatment of a subset of patients who experience a high burden of disease; this study found that if patient groups examined in the study were assessed by objective severity measures alone they would have been classified as having mild to moderate disease although their DLQIs indicated high impact or severe disease.

It is becoming accepted by the dermatology medical community and patients with psoriasis that systemic therapy with highly efficacious biologic medications should be considered even in situations where traditional scoring systems indicate mild to moderate disease severity. To address this unmet need, numerous sponsors and academic researchers have conducted clinical studies to evaluate the efficacy and safety of advanced, systemic therapies in patients with lower BSA involvement psoriasis.

Consequently, patients with mild to moderate psoriasis represent an underserved population. There remains an unmet medical need for effective, convenient, and safe treatment of mild to moderate psoriasis that is well tolerated compared to currently available treatment options

### SUMMARY OF THE INVENTION

In a first aspect, the invention concerns a method of treating a subject (patient) suffering from mild to moderate psoriasis comprising administering an anti-IL23 specific antibody (also referred to as IL23p19 or IL23p19 subunit antibody), e.g., guselkumab, to the patient in an initial dose at the start of treatment and a time interval thereafter, preferably, an additional dose 4 weeks after the initial dose and then a dose about every 8 weeks after the additional dose, e.g., a dose at 0, 4, 12, 20, 28, 36, 44 and/or 52 weeks. In addition, in another embodiment the treatment continues through 100 weeks or longer after the start of treatment.

In one embodiment, the subject receives the anti-IL23 specific antibody subcutaneously at a dose of 100 mg in the initial dose, the additional dose 4 weeks after the initial dose and every 8 weeks after the additional dose.

In another aspect, the composition used in the method of the invention comprises a pharmaceutical composition comprising: an anti-IL23 specific antibody.

In an embodiment, mild to moderate psoriasis patients achieve significant improvement in clinical and/or exploratory endpoints selected from the group consisting of:
(i) Achievement of an IGA score of cleared (0) or minimal (1) with at least ≥2 grade improvement from baseline at Week 16;
(ii) Achievement of BSA≤1% at Week 16;
(iii) Achievement of an IGA score of cleared (0) at Week 16;
(iv) Achievement of a PASI 90 response at Week 16;
(v) Achievement of a PASI 100 response at Week 16;
(vi) Achievement of a Scalp-Specific Investigator's Global Assessment (ss-IGA) score of absence of disease (0) or very mild disease and have at least a 2-grade improvement from baseline at Week 16 and an ss-IGA score ≥2 at baseline;
(vii) Achievement of ≥4-point reduction (improvement) in Psoriasis Symptom and Sign Diary (PSSD) Itch score from baseline at Week 16 among participants with a PSSD Itch score ≥4 at baseline;
(viii) Achievement of BSA≤1% at Week 16;
(ix) Achievement of an IGA score of cleared (0) at Week 16;
(x) Achievement of a PASI 90 response at Week 16;
(xi) Achievement of a PASI 100 response at Week 16;
(xii) Achievement of a ss-IGA score of absence of disease (0) or very mild disease and have at least a 2-grade improvement from baseline at Week 16 among randomized participants with scalp psoriasis and an ss-IGA score ≥2 at baseline;
(xiii) Achievement of a PSSD symptom score of 0 at Week 24 vs apremilast, among randomized participants with a baseline PSSD symptom score ≥1;
(xiv) Achievement of ≥4-point reduction (improvement) in PSSD Itch score from baseline at Week 16 among participants with a PSSD Itch score ≥4 at baseline; and
(xv) Percent improvement from baseline in the Nail Psoriasis Severity Index (NAPSI) at Week 16 among randomized participants with nail psoriasis at baseline.

In preferred embodiments, the endpoints may be measured about 16, 24, 48 or more weeks after initial treatment.

In another aspect of the invention the pharmaceutical composition comprises an isolated anti-IL23 specific antibody having the CDR sequences comprising (i) the heavy chain CDR amino acid sequences of SEQ ID NO: 1, SEQ ID NO: 2, and SEQ ID NO: 3; and (ii) the light chain CDR amino acid sequences of SEQ ID NO: 4, SEQ ID NO: 5, and SEQ ID NO: 6, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

Another aspect of the method of the invention comprises administering a pharmaceutical composition comprising an isolated anti-IL-23 specific antibody having the heavy chain variable region amino acid sequence of SEQ ID NO: 7 and the light chain variable region amino acid sequence of SEQ ID NO: 8, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state for use in the treatment of a patient with mild to moderate psoriasis according to the doses and dosing regimens described herein.

A further aspect of the method of the invention comprises administering a pharmaceutical composition comprising an isolated anti-IL-23 specific antibody having the heavy chain amino acid sequence of SEQ ID NO: 9 and the light chain amino acid sequence of SEQ ID NO: 10, optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

In a still further embodiment, the method of the invention comprises administering a pharmaceutical composition comprising the antibody guselkumab (marketed by Janssen Biotech, Inc as Tremfya®), optionally in a composition of 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

The details of one or more embodiments of the invention are set forth in the description below. Other features and advantages will be apparent from the following detailed description, figures, and the appended claims.

### BRIEF DESCRIPTION OF THE DRAWINGS

In the Figures:

FIG. 1 shows a schematic of the clinical trial design described below.

### DETAILED DESCRIPTION OF THE PREFERRED EMBODIMENTS

As used herein the method of treatment of a subject suffering from mild to moderate psoriasis comprises administering isolated, recombinant and/or synthetic anti-IL-23 specific human antibodies and diagnostic and therapeutic compositions, methods and devices.

As used herein, an “anti-IL-23 specific antibody,” “anti-IL-23 antibody,” “antibody portion,” or “antibody fragment” and/or “antibody variant” and the like include any protein or peptide containing molecule that comprises at least a portion of an immunoglobulin molecule, such as but not limited to, at least one complementarity determining region (CDR) of a heavy or light chain or a ligand binding portion thereof, a heavy chain or light chain variable region, a heavy chain or light chain constant region, a framework region, or any portion thereof, or at least one portion of an IL-23 receptor or binding protein, which can be incorporated into an antibody of the present invention. Such antibody optionally further affects a specific ligand, such as but not limited to, where such antibody modulates, decreases, increases, antagonizes, agonizes, mitigates, alleviates, blocks, inhibits, abrogates and/or interferes with at least one IL-23 activity or binding, or with IL-23 receptor activity or binding, in vitro, in situ and/or in vivo. As a non-limiting example, a suitable anti-IL-23 antibody, specified portion or variant of the present invention can bind at least one IL-23 molecule, or specified portions, variants or domains thereof. A suitable anti-IL-23 antibody, specified portion, or variant can also optionally affect at least one of IL-23 activity or function, such as but not limited to, RNA, DNA or protein synthesis, IL-23 release, IL-23 receptor signaling, membrane IL-23 cleavage, IL-23 activity, IL-23 production and/or synthesis.

The term “antibody” is further intended to encompass antibodies, digestion fragments, specified portions and variants thereof, including antibody mimetics or comprising portions of antibodies that mimic the structure and/or function of an antibody or specified fragment or portion thereof, including single chain antibodies and fragments thereof. Functional fragments include antigen-binding fragments that bind to a mammalian IL-23. For example, antibody fragments capable of binding to IL-23 or portions thereof, including, but not limited to, Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Antibodies can also be produced in a variety of truncated forms using antibody genes in which one or more stop codons have been introduced upstream of the natural stop site. For example, a combination gene encoding a F(ab′)₂ heavy chain portion can be designed to include DNA sequences encoding the C_{H}1 domain and/or hinge region of the heavy chain. The various portions of antibodies can be joined together chemically by conventional techniques or can be prepared as a contiguous protein using genetic engineering techniques.

As used herein, the term “human antibody” refers to an antibody in which substantially every part of the protein (e.g., CDR, framework, C_{L}, C_{H} domains (e.g., C_{H}1, C_{H}2, C_{H}3), hinge, (V_{L}, V_{H})) is substantially non-immunogenic in humans, with only minor sequence changes or variations. A “human antibody” may also be an antibody that is derived from or closely matches human germline immunoglobulin sequences. Human antibodies may include amino acid residues not encoded by germline immunoglobulin sequences (e.g., mutations introduced by random or site-specific mutagenesis in vitro or by somatic mutation in vivo). Often, this means that the human antibody is substantially non-immunogenic in humans. Human antibodies have been classified into groupings based on their amino acid sequence similarities. Accordingly, using a sequence similarity search, an antibody with a similar linear sequence can be chosen as a template to create a human antibody. Similarly, antibodies designated primate (monkey, baboon, chimpanzee, etc.), rodent (mouse, rat, rabbit, guinea pig, hamster, and the like) and other mammals designate such species, sub-genus, genus, sub-family, and family specific antibodies. Further, chimeric antibodies can include any combination of the above. Such changes or variations optionally and preferably retain or reduce the immunogenicity in humans or other species relative to non-modified antibodies. Thus, a human antibody is distinct from a chimeric or humanized antibody.

It is pointed out that a human antibody can be produced by a non-human animal or prokaryotic or eukaryotic cell that is capable of expressing functionally rearranged human immunoglobulin (e.g., heavy chain and/or light chain) genes. Further, when a human antibody is a single chain antibody, it can comprise a linker peptide that is not found in native human antibodies. For example, an Fv can comprise a linker peptide, such as two to about eight glycine or other amino acid residues, which connects the variable region of the heavy chain and the variable region of the light chain. Such linker peptides are considered to be of human origin.

Bispecific, heterospecific, heteroconjugate or similar antibodies can also be used that are monoclonal, preferably, human or humanized, antibodies that have binding specificities for at least two different antigens. In the present case, one of the binding specificities is for at least one IL-23 protein, the other one is for any other antigen. Methods for making bispecific antibodies are known in the art. Traditionally, the recombinant production of bispecific antibodies is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed, e.g., in WO 93/08829, U.S. Pat. Nos. 6,210,668, 6,193,967, 6,132,992, 6,106,833, 6,060,285, 6,037,453, 6,010,902, 5,989,530, 5,959,084, 5,959,083, 5,932,448, 5,833,985, 5,821,333, 5,807,706, 5,643,759, 5,601,819, 5,582,996, 5,496,549, 4,676,980, WO 91/00360, WO 92/00373, EP 03089, Traunecker et al., EMBO J. 10:3655 (1991), Suresh et al., Methods in Enzymology 121:210 (1986), each entirely incorporated herein by reference.

Anti-IL-23 specific (also termed IL-23 specific antibodies) (or antibodies to IL-23) useful in the methods and compositions of the present invention can optionally be characterized by high affinity binding to IL-23 and, optionally and preferably, having low toxicity. In particular, an antibody, specified fragment or variant of the invention, where the individual components, such as the variable region, constant region and framework, individually and/or collectively, optionally and preferably possess low immunogenicity, is useful in the present invention. The antibodies that can be used in the invention are optionally characterized by their ability to treat patients for extended periods with measurable alleviation of symptoms and low and/or acceptable toxicity. Low or acceptable immunogenicity and/or high affinity, as well as other suitable properties, can contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference). “Low immunogenicity” can also be defined as the incidence of titrable levels of antibodies to the anti-IL-23 antibody in patients treated with anti-IL-23 antibody as occurring in less than 25% of patients treated, preferably, in less than 10% of patients treated with the recommended dose for the recommended course of therapy during the treatment period.

The term “safe,” as it relates to a dose, dosage regimen, treatment or method with an anti-IL-23 antibody of the present invention (e.g., the anti-IL-23 antibody guselkumab), refers to a relatively low or reduced frequency and/or low or reduced severity of treatment-emergent adverse events (referred to as AEs or TEAEs) from the clinical trials conducted, e.g., Phase 2 clinical trials and earlier, compared to the standard of care or to another comparator. An adverse event is an untoward medical occurrence in a patient administered a medicinal product. In particular, safe as it relates to a dose, dosage regimen or treatment with an anti-IL-23 antibody of the present invention refers to a relatively low or reduced frequency and/or low or reduced severity of adverse events associated with administration of the antibody if attribution is considered to be possible, probable, or very likely due to the use of the anti-IL-23 antibody.

Utility

The isolated nucleic acids of the present invention can be used for production of at least one anti-IL-23 antibody or specified variant thereof, which can be used to measure or effect in a cell, tissue, organ or animal (including mammals and humans), to diagnose, monitor, modulate, treat, alleviate, help prevent the incidence of, or reduce the symptoms of mild to moderate psoriasis.

Such a method can comprise administering an effective amount of a composition or a pharmaceutical composition comprising at least one anti-IL-23 antibody to a cell, tissue, organ, animal or patient in need of such modulation, treatment, alleviation, prevention, or reduction in symptoms, effects or mechanisms. The effective amount can comprise an amount of about 0.001 to 500 mg/kg per single (e.g., bolus), multiple or continuous administration, or to achieve a serum concentration of 0.01-5000 μg/ml serum concentration per single, multiple, or continuous administration, or any effective range or value therein, as done and determined using known methods, as described herein or known in the relevant arts.

Citations

All publications or patents cited herein, whether or not specifically designated, are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, NY (1987-2001); Sambrook, et al., Molecular Cloning: A Laboratory Manual, 2nd Edition, Cold Spring Harbor, N Y (1989); Harlow and Lane, antibodies, a Laboratory Manual, Cold Spring Harbor, N Y (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001).

Antibodies of the Present Invention—Production and Generation

At least one anti-IL-23 antibody used in the method of the present invention can be optionally produced by a cell line, a mixed cell line, an immortalized cell or clonal population of immortalized cells, as well known in the art. See, e.g., Ausubel, et al., ed., Current Protocols in Molecular Biology, John Wiley & Sons, Inc., NY, NY (1987-2001); Sambrook, et al., Molecular Cloning: A Laboratory Manual, 2^{nd} Edition, Cold Spring Harbor, N Y (1989); Harlow and Lane, antibodies, a Laboratory Manual, Cold Spring Harbor, N Y (1989); Colligan, et al., eds., Current Protocols in Immunology, John Wiley & Sons, Inc., NY (1994-2001); Colligan et al., Current Protocols in Protein Science, John Wiley & Sons, NY, NY, (1997-2001), each entirely incorporated herein by reference.

A preferred anti-IL-23 antibody is guselkumab (also referred to as CNTO1959) having the heavy chain amino acid sequence of SEQ ID NO: 9 and the light chain amino acid sequence of SEQ ID NO: 10; having the heavy chain variable region amino acid sequence of SEQ ID NO: 7 and the light chain variable region amino acid sequence of SEQ ID NO: 8; and having the heavy chain CDR amino acid sequences of SEQ ID NO: 1, SEQ ID NO: 2, and SEQ ID NO: 3; and the light chain CDR amino acid sequences of SEQ ID NO: 4, SEQ ID NO: 5, and SEQ ID NO: 6. Other anti-IL-23 antibodies have sequences listed herein and are described in U.S. Pat. No. 7,935,344, the entire contents of which are incorporated herein by reference).

Human antibodies that are specific for human IL-23 proteins or fragments thereof can be raised against an appropriate immunogenic antigen, such as an isolated IL-23 protein and/or a portion thereof (including synthetic molecules, such as synthetic peptides). Other specific or general mammalian antibodies can be similarly raised. Preparation of immunogenic antigens, and monoclonal antibody production can be performed using any suitable technique.

In one approach, a hybridoma is produced by fusing a suitable immortal cell line (e.g., a myeloma cell line, such as, but not limited to, Sp2/0, Sp2/0-AG14, NSO, NS1, NS2, AE-1, L.5, L243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMALWA, NEURO 2A, or the like, or heteromylomas, fusion products thereof, or any cell or fusion cell derived therefrom, or any other suitable cell line as known in the art) (see, e.g., www.atcc.org, www.lifetech.com, and the like), with antibody producing cells, such as, but not limited to, isolated or cloned spleen, peripheral blood, lymph, tonsil, or other immune or B cell containing cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Antibody producing cells can also be obtained from the peripheral blood or, preferably, the spleen or lymph nodes, of humans or other suitable animals that have been immunized with the antigen of interest. Any other suitable host cell can also be used for expressing heterologous or endogenous nucleic acid encoding an antibody, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce antibodies with the desired specificity can be selected by a suitable assay (e.g., ELISA).

Other suitable methods of producing or isolating antibodies of the requisite specificity can be used, including, but not limited to, methods that select recombinant antibody from a peptide or protein library (e.g., but not limited to, a bacteriophage, ribosome, oligonucleotide, RNA, cDNA, or the like, display library; e.g., as available from Cambridge antibody Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, CA; Ixsys. See, e.g., EP 368,684, PCT/GB91/01134; PCT/GB92/01755; PCT/GB92/002240; PCT/GB92/00883; PCT/GB93/00605; U.S. Ser. No. 08/350,260 (May 12, 1994); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); WO96/13583, WO97/08320 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,817,483, 5,824,514, 5,976,862, WO 86/05803, EP 590 689 (Ixsys, predecessor of Applied Molecular Evolution (AME), each entirely incorporated herein by reference)) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and applications) that are capable of producing a repertoire of human antibodies, as known in the art and/or as described herein. Such techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell antibody producing technologies (e.g., selected lymphocyte antibody method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, MA; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for engineering or humanizing non-human or human antibodies can also be used and are well known in the art. Generally, a humanized or engineered antibody has one or more amino acid residues from a source that is non-human, e.g., but not limited to, mouse, rat, rabbit, non-human primate or other mammal. These non-human amino acid residues are replaced by residues often referred to as “import” residues, which are typically taken from an “import” variable, constant or other domain of a known human sequence.

Such imported sequences can be used to reduce immunogenicity or reduce, enhance or modify binding, affinity, on-rate, off-rate, avidity, specificity, half-life, or any other suitable characteristic, as known in the art. In general, the CDR residues are directly and most substantially involved in influencing antigen binding. Accordingly, part or all of the non-human or human CDR sequences are maintained while the non-human sequences of the variable and constant regions may be replaced with human or other amino acids.

Antibodies can also optionally be humanized or human antibodies engineered with retention of high affinity for the antigen and other favorable biological properties. To achieve this goal, humanized (or human) antibodies can be optionally prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved.

In addition, the human IL-23 specific antibody used in the method of the present invention may comprise a human germline light chain framework. In particular embodiments, the light chain germline sequence is selected from human VK sequences including, but not limited to, A1, A10, A11, A14, A17, A18, A19, A2, A20, A23, A26, A27, A3, A30, A5, A7, B2, B3, L1, L10, L11, L12, L14, L15, L16, L18, L19, L2, L20, L22, L23, L24, L25, L4/18a, L5, L6, L8, L9, O1, O11, O12, O14, O18, O2, O4, and O8. In certain embodiments, this light chain human germline framework is selected from V1-11, V1-13, V1-16, V1-17, V1-18, V1-19, V1-2, V1-20, V1-22, V1-3, V1-4, V1-5, V1-7, V1-9, V2-1, V2-11, V2-13, V2-14, V2-15, V2-17, V2-19, V2-6, V2-7, V2-8, V3-2, V3-3, V3-4, V4-1, V4-2, V4-3, V4-4, V4-6, V5-1, V5-2, V5-4, and V5-6.

In other embodiments, the human IL-23 specific antibody used in the method of the present invention may comprise a human germline heavy chain framework. In particular embodiments, this heavy chain human germline framework is selected from VH1-18, VH1-2, VH1-24, VH1-3, VH1-45, VH1-46, VH1-58, VH1-69, VH1-8, VH2-26, VH2-5, VH2-70, VH3-11, VH3-13, VH3-15, VH3-16, VH3-20, VH3-21, VH3-23, VH3-30, VH3-33, VH3-35, VH3-38, VH3-43, VH3-48, VH3-49, VH3-53, VH3-64, VH3-66, VH3-7, VH3-72, VH3-73, VH3-74, VH3-9, VH4-28, VH4-31, VH4-34, VH4-39, VH4-4, VH4-59, VH4-61, VH5-51, VH6-1, and VH7-81.

In particular embodiments, the light chain variable region and/or heavy chain variable region comprises a framework region or at least a portion of a framework region (e.g., containing 2 or 3 subregions, such as FR2 and FR3). In certain embodiments, at least FRL1, FRL2, FRL3, or FRL4 is fully human. In other embodiments, at least FRH1, FRH2, FRH3, or FRH4 is fully human. In some embodiments, at least FRL1, FRL2, FRL3, or FRL4 is a germline sequence (e.g., human germline) or comprises human consensus sequences for the particular framework (readily available at the sources of known human Ig sequences described above). In other embodiments, at least FRH1, FRH2, FRH3, or FRH4 is a germline sequence (e.g., human germline) or comprises human consensus sequences for the particular framework. In preferred embodiments, the framework region is a fully human framework region.

Humanization or engineering of antibodies of the present invention can be performed using any known method, such as but not limited to those described in, Winter (Jones et al., Nature 321:522 (1986); Riechmann et al., Nature 332:323 (1988); Verhoeyen et al., Science 239:1534 (1988)), Sims et al., J. Immunol. 151: 2296 (1993); Chothia and Lesk, J. Mol. Biol. 196:901 (1987), Carter et al., Proc. Natl. Acad. Sci. U.S.A. 89:4285 (1992); Presta et al., J. Immunol. 151:2623 (1993), U.S. Pat. Nos. 5,723,323, 5,976,862, 5,824,514, 5,817,483, 5,814,476, 5,763,192, 5,723,323, 5,766886, 5714352, 6204023, 6180370, 5693762, 5530101, 5585089, 5225539; 4816567, PCT/: US98/16280, US96/18978, US91/09630, US91/05939, US94/01234, GB89/01334, GB91/01134, GB92/01755; WO90/14443, WO90/14424, WO90/14430, EP 229246, each entirely incorporated herein by reference, included references cited therein.

In certain embodiments, the antibody comprises an altered (e.g., mutated) Fc region. For example, in some embodiments, the Fc region has been altered to reduce or enhance the effector functions of the antibody. In some embodiments, the Fc region is an isotype selected from IgM, IgA, IgG, IgE, or other isotype. Alternatively or additionally, it may be useful to combine amino acid modifications with one or more further amino acid modifications that alter C1q binding and/or the complement dependent cytotoxicity function of the Fc region of an IL-23 binding molecule. The starting polypeptide of particular interest may be one that binds to C1q and displays complement dependent cytotoxicity (CDC). Polypeptides with pre-existing C1q binding activity, optionally further having the ability to mediate CDC may be modified such that one or both of these activities are enhanced. Amino acid modifications that alter C1q and/or modify its complement dependent cytotoxicity function are described, for example, in WO0042072, which is hereby incorporated by reference.

As disclosed above, one can design an Fc region of the human IL-23 specific antibody of the present invention with altered effector function, e.g., by modifying C1q binding and/or FcγR binding and thereby changing complement dependent cytotoxicity (CDC) activity and/or antibody-dependent cell-mediated cytotoxicity (ADCC) activity. “Effector functions” are responsible for activating or diminishing a biological activity (e.g., in a subject). Examples of effector functions include, but are not limited to: C1q binding; CDC; Fc receptor binding; ADCC; phagocytosis; down regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of the human IL-23 (or anti-IL-23) antibody with improved C1q binding and improved FcγRIIIbinding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

Fc mutations can also be introduced in engineer to alter their interaction with the neonatal Fc receptor (FcRn) and improve their pharmacokinetic properties. A collection of human Fc variants with improved binding to the FcRn have been described (Shields et al., (2001). High resolution mapping of the binding site on human IgG1 for FcγRI, FcγRII, FcγRIII, and FcRn and design of IgG1 variants with improved binding to the FcγR, J. Biol. Chem. 276:6591-6604).

Another type of amino acid substitution serves to alter the glycosylation pattern of the Fc region of the human IL-23 specific antibody. Glycosylation of an Fc region is typically either N-linked or O-linked. N-linked refers to the attachment of the carbohydrate moiety to the side chain of an asparagine residue. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used. The recognition sequences for enzymatic attachment of the carbohydrate moiety to the asparagine side chain peptide sequences are asparagine-X-serine and asparagine-X-threonine, where X is any amino acid except proline. Thus, the presence of either of these peptide sequences in a polypeptide creates a potential glycosylation site.

The glycosylation pattern may be altered, for example, by deleting one or more glycosylation site(s) found in the polypeptide, and/or adding one or more glycosylation sites that are not present in the polypeptide. Addition of glycosylation sites to the Fc region of a human IL-23 specific antibody is conveniently accomplished by altering the amino acid sequence such that it contains one or more of the above-described tripeptide sequences (for N-linked glycosylation sites). An exemplary glycosylation variant has an amino acid substitution of residue Asn 297 of the heavy chain. The alteration may also be made by the addition of, or substitution by, one or more serine or threonine residues to the sequence of the original polypeptide (for O-linked glycosylation sites). Additionally, a change of Asn 297 to Ala can remove one of the glycosylation sites.

In certain embodiments, the human IL-23 specific antibody of the present invention is expressed in cells that express beta (1,4)-N-acetylglucosaminyltransferase III (GnT III), such that GnT III adds GlcNAc to the human IL-23 antibody. Methods for producing antibodies in such a fashion are provided in WO/9954342, WO/03011878, patent publication 20030003097A1, and Umana et al., Nature Biotechnology, 17:176-180, February 1999; all of which are herein specifically incorporated by reference in their entireties.

The anti-IL-23 antibody can also be optionally generated by immunization of a transgenic animal (e.g., mouse, rat, hamster, non-human primate, and the like) capable of producing a repertoire of human antibodies, as described herein and/or as known in the art. Cells that produce a human anti-IL-23 antibody can be isolated from such animals and immortalized using suitable methods, such as the methods described herein.

Transgenic mice that can produce a repertoire of human antibodies that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B1, Kucherlapate et al. EP 0710 719 A1, Surani et al. U.S. Pat. No. 5,545,807, Bruggemann et al. WO 90/04036, Bruggemann et al. EP 0438 474 B1, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(1):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce antibodies encoded by endogenous genes.

Screening antibodies for specific binding to similar proteins or fragments can be conveniently achieved using peptide display libraries. This method involves the screening of large collections of peptides for individual members having the desired function or structure. Antibody screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980, 91/19818, and 93/08278.

Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, CA), and Cambridge antibody Technologies (Cambridgeshire, UK). See, e.g., U.S. Pat. Nos. 4,704,692, 4,939,666, 4,946,778, 5,260,203, 5,455,030, 5,518,889, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, 5427908, 5580717, assigned to Affymax; 5885793, assigned to Cambridge antibody Technologies; 5750373, assigned to Genentech, 5618920, 5595898, 5576195, 5698435, 5693493, 5698417, assigned to Xoma, Colligan, supra; Ausubel, supra; or Sambrook, supra, each of the above patents and publications entirely incorporated herein by reference.

Antibodies used in the method of the present invention can also be prepared using at least one anti-IL23 antibody encoding nucleic acid to provide transgenic animals or mammals, such as goats, cows, horses, sheep, rabbits, and the like, that produce such antibodies in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

The antibodies used in the method of the invention can bind human IL-23 with a wide range of affinities (K_{D}). In a preferred embodiment, a human mAb can optionally bind human IL-23 with high affinity. For example, a human mAb can bind human IL-23 with a K_{D} equal to or less than about 10⁻⁷ M, such as but not limited to, 0.1-9.9 (or any range or value therein)×10⁻⁷, 10⁻⁸, 10⁻⁹, 10⁻¹⁰, 10⁻¹¹, 10⁻¹², 10⁻¹³ or any range or value therein.

The affinity or avidity of an antibody for an antigen can be determined experimentally using any suitable method. (See, for example, Berzofsky, et al., “Antibody-Antigen Interactions,” In Fundamental Immunology, Paul, W. E., Ed., Raven Press: New York, NY (1984); Kuby, Janis Immunology, W. H. Freeman and Company: New York, NY (1992); and methods described herein). The measured affinity of a particular antibody-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of antibody and antigen, and a standardized buffer, such as the buffer described herein.

Nucleic Acid Molecules

Using the information provided herein, for example, the nucleotide sequences encoding at least 70-100% of the contiguous amino acids of at least one of the light or heavy chain variable or CDR regions described herein, among other sequences disclosed herein, specified fragments, variants or consensus sequences thereof, or a deposited vector comprising at least one of these sequences, a nucleic acid molecule of the present invention encoding at least one anti-IL-23 antibody can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules used in the method of the present invention can include nucleic acid molecules comprising an open reading frame (ORF), optionally, with one or more introns, e.g., but not limited to, at least one specified portion of at least one CDR, such as CDR1, CDR2 and/or CDR3 of at least one heavy chain or light chain; nucleic acid molecules comprising the coding sequence for an anti-IL-23 antibody or variable region; and nucleic acid molecules which comprise a nucleotide sequence substantially different from those described above but which, due to the degeneracy of the genetic code, still encode at least one anti-IL-23 antibody as described herein and/or as known in the art. Of course, the genetic code is well known in the art. Thus, it would be routine for one skilled in the art to generate such degenerate nucleic acid variants that code for specific anti-IL-23 antibodies used in the method of the present invention. See, e.g., Ausubel, et al., supra, and such nucleic acid variants are included in the present invention. Non-limiting examples of isolated nucleic acid molecules include nucleic acids encoding HC CDR1, HC CDR2, HC CDR3, LC CDR1, LC CDR2, and LC CDR3, respectively.

As indicated herein, nucleic acid molecules which comprise a nucleic acid encoding an anti-IL-23 antibody can include, but are not limited to, those encoding the amino acid sequence of an antibody fragment, by itself; the coding sequence for the entire antibody or a portion thereof; the coding sequence for an antibody, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example, ribosome binding and stability of mRNA); an additional coding sequence that codes for additional amino acids, such as those that provide additional functionalities. Thus, the sequence encoding an antibody can be fused to a marker sequence, such as a sequence encoding a peptide that facilitates purification of the fused antibody comprising an antibody fragment or portion.

Polynucleotides Selectively Hybridizing to a Polynucleotide as Described Herein

The method of the present invention uses isolated nucleic acids that hybridize under selective hybridization conditions to a polynucleotide disclosed herein. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Optionally, polynucleotides will encode at least a portion of an antibody. The polynucleotides embrace nucleic acid sequences that can be employed for selective hybridization to a polynucleotide encoding an antibody of the present invention. See, e.g., Ausubel, supra; Colligan, supra, each entirely incorporated herein by reference.

Construction of Nucleic Acids

The isolated nucleic acids can be made using (a) recombinant methods, (b) synthetic techniques, (c) purification techniques, and/or (d) combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention, excluding the coding sequence, is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

Recombinant Methods for Constructing Nucleic Acids

The isolated nucleic acid compositions, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, are well known to those of ordinary skill in the art. (See, e.g., Ausubel, supra; or Sambrook, supra)

Recombinant Expression Cassettes

The present invention uses recombinant expression cassettes comprising a nucleic acid. A nucleic acid sequence, for example, a cDNA or a genomic sequence encoding an antibody used in the method of the present invention, can be used to construct a recombinant expression cassette that can be introduced into at least one desired host cell. A recombinant expression cassette will typically comprise a polynucleotide operably linked to transcriptional initiation regulatory sequences that will direct the transcription of the polynucleotide in the intended host cell. Both heterologous and non-heterologous (i.e., endogenous) promoters can be employed to direct expression of the nucleic acids.

In some embodiments, isolated nucleic acids that serve as promoter, enhancer, or other elements can be introduced in the appropriate position (upstream, downstream or in the intron) of a non-heterologous form of a polynucleotide of the present invention so as to up or down regulate expression of a polynucleotide. For example, endogenous promoters can be altered in vivo or in vitro by mutation, deletion and/or substitution.

Vectors and Host Cells

The present invention also relates to vectors that include isolated nucleic acid molecules, host cells that are genetically engineered with the recombinant vectors, and the production of at least one anti-IL-23 antibody by recombinant techniques, as is well known in the art. See, e.g., Sambrook, et al., supra; Ausubel, et al., supra, each entirely incorporated herein by reference.

Purification of an Antibody

An anti-IL-23 antibody can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See, e.g., Colligan, Current Protocols in Immunology, or Current Protocols in Protein Science, John Wiley & Sons, NY, NY, (1997-2001), e.g., Chapters 1, 4, 6, 8, 9, 10, each entirely incorporated herein by reference.

Antibodies used in the method of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the antibody can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

Anti-IL-23 Antibodies.

An anti-IL-23 antibody according to the present invention includes any protein or peptide containing molecule that comprises at least a portion of an immunoglobulin molecule, such as but not limited to, at least one ligand binding portion (LBP), such as but not limited to, a complementarity determining region (CDR) of a heavy or light chain or a ligand binding portion thereof, a heavy chain or light chain variable region, a framework region (e.g., FR1, FR2, FR3, FR4 or fragment thereof, further optionally comprising at least one substitution, insertion or deletion), a heavy chain or light chain constant region, (e.g., comprising at least one C_{H}1, hinge1, hinge2, hinge3, hinge4, C_{H}2, or C_{H}3 or fragment thereof, further optionally comprising at least one substitution, insertion or deletion), or any portion thereof, that can be incorporated into an antibody. An antibody can include or be derived from any mammal, such as but not limited to, a human, a mouse, a rabbit, a rat, a rodent, a primate, or any combination thereof, and the like.

The isolated antibodies used in the method of the present invention comprise the antibody amino acid sequences disclosed herein encoded by any suitable polynucleotide, or any isolated or prepared antibody. Preferably, the human antibody or antigen-binding fragment binds human IL-23 and, thereby, partially or substantially neutralizes at least one biological activity of the protein. An antibody, or specified portion or variant thereof, that partially or preferably substantially neutralizes at least one biological activity of at least one IL-23 protein or fragment can bind the protein or fragment and thereby inhibit activities mediated through the binding of IL-23 to the IL-23 receptor or through other IL-23-dependent or mediated mechanisms. As used herein, the term “neutralizing antibody” refers to an antibody that can inhibit an IL-23-dependent activity by about 20-120%, preferably by at least about 10, 20, 30, 40, 50, 55, 60, 65, 70, 75, 80, 85, 90, 91, 92, 93, 94, 95, 96, 97, 98, 99, 100% or more depending on the assay. The capacity of an anti-IL-23 antibody to inhibit an IL-23-dependent activity is preferably assessed by at least one suitable IL-23 protein or receptor assay, as described herein and/or as known in the art. A human antibody can be of any class (IgG, IgA, IgM, IgE, IgD, etc.) or isotype and can comprise a kappa or lambda light chain. In one embodiment, the human antibody comprises an IgG heavy chain or defined fragment, for example, at least one of isotypes, IgG1, IgG2, IgG3 or IgG4 (e.g., γ1, γ2, γ3, γ4). Antibodies of this type can be prepared by employing a transgenic mouse or other transgenic non-human mammal comprising at least one human light chain (e.g., IgG, IgA, and IgM) transgenes as described herein and/or as known in the art. In another embodiment, the anti-IL-23 human antibody comprises an IgG1 heavy chain and an IgG1 light chain.

An antibody binds at least one specified epitope specific to at least one IL-23 protein, subunit, fragment, portion or any combination thereof. The at least one epitope can comprise at least one antibody binding region that comprises at least one portion of the protein, which epitope is preferably comprised of at least one extracellular, soluble, hydrophillic, external or cytoplasmic portion of the protein.

Generally, the human antibody or antigen-binding fragment will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. The CDR sequences may be derived from human germline sequences or closely match the germline sequences. For example, the CDRs from a synthetic library derived from the original non-human CDRs can be used. These CDRs may be formed by incorporation of conservative substitutions from the original non-human sequence. In another particular embodiment, the antibody or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3.

Such antibodies can be prepared by chemically joining together the various portions (e.g., CDRs, framework) of the antibody using conventional techniques, by preparing and expressing a (i.e., one or more) nucleic acid molecule that encodes the antibody using conventional techniques of recombinant DNA technology or by using any other suitable method.

The anti-IL-23 specific antibody can comprise at least one of a heavy or light chain variable region having a defined amino acid sequence. For example, in a preferred embodiment, the anti-IL-23 antibody comprises at least one of a heavy chain variable region, optionally having the amino acid sequence of SEQ ID NO:7 and/or at least one light chain variable region, optionally having the amino acid sequence of SEQ ID NO:8. In an additional preferred embodiment, the anti-IL-23 antibody comprises at least one heavy chain, optionally having the amino acid sequence of SEQ ID NO:9 and/or at least one light chain, optionally having the amino acid sequence of SEQ ID NO:10. Antibodies that bind to human IL-23 and that comprise a defined heavy or light chain variable region can be prepared using suitable methods, such as phage display (Katsube, Y., et al., Int J Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human IL-23 or a fragment thereof to elicit the production of antibodies. If desired, the antibody producing cells can be isolated and hybridomas or other immortalized antibody-producing cells can be prepared as described herein and/or as known in the art. Alternatively, the antibody, specified portion or variant can be expressed using the encoding nucleic acid or portion thereof in a suitable host cell.

The invention also relates to antibodies, antigen-binding fragments, immunoglobulin chains and CDRs comprising amino acids in a sequence that is substantially the same as an amino acid sequence described herein. Preferably, such antibodies or antigen-binding fragments and antibodies comprising such chains or CDRs can bind human IL-23 with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g., charge, structure, polarity, hydrophobicity/hydrophilicity) that are similar to those of the first amino acid. Conservative substitutions include, without limitation, replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Amino Acid Codes

The amino acids that make up anti-IL-23 antibodies of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE | THREE | | THREE |
|---|---|---|---|
| LETTER | LETTER | | NUCLEOTIDE |
| CODE | CODE | NAME | CODON(S) |
| A | Ala | Alanine | GCA, GCC, GCG, |
| | | | GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, |
| | | | GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, |
| | | | CUC, CUG, CUU |
| M | Met | Methionine | AUG |
| N | Asn | Asparagine | AAC, AAU |
| P | Pro | Proline | CCA, CCC, CCG, |
| | | | CCU |
| Q | Gln | Glutamine | CAA, CAG |
| R | Arg | Arginine | AGA, AGG, CGA, |
| | | | CGC, CGG, CGU |
| S | Ser | Serine | AGC, AGU, UCA, |
| | | | UCC, UCG, UCU |
| T | Thr | Threonine | ACA, ACC, ACG, |
| | | | ACU |
| V | Val | Valine | GUA, GUC, GUG, |
| | | | GUU |
| W | Trp | Tryptophan | UGG |
| Y | Tyr | Tyrosine | UAC, UAU |

An anti-IL-23 antibody used in the method of the present invention can include one or more amino acid substitutions, deletions or additions, either from natural mutations or human manipulation, as specified herein.

Anti-IL-23 antibodies can include, but are not limited to, at least one portion, sequence or combination selected from 5 to all of the contiguous amino acids of at least one of SEQ ID NOS: 1, 2, 3, 4, 5, and 6.

IL-23 antibodies or specified portions or variants can include, but are not limited to, at least one portion, sequence or combination selected from at least 3-5 contiguous amino acids of the SEQ ID NOs above; 5-17 contiguous amino acids of the SEQ ID NOs above, 5-10 contiguous amino acids of the SEQ ID NOs above, 5-11 contiguous amino acids of the SEQ ID NOs above, 5-7 contiguous amino acids of the SEQ ID NOs above; 5-9 contiguous amino acids of the SEQ ID NOs above.

An anti-IL-23 antibody can further optionally comprise a polypeptide of at least one of 70-100% of the SEQ ID NOs above. In one embodiment, the amino acid sequence of an immunoglobulin chain, or portion thereof (e.g., variable region, CDR) has about 70-100% identity (e.g., 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 or any range or value therein) to the amino acid sequence of the corresponding chain of at least one of the SEQ ID NOs above. For example, the amino acid sequence of a light chain variable region can be compared with the sequence of the SEQ ID NOs above, or the amino acid sequence of a heavy chain CDR3 can be compared with the SEQ ID NOs above. Preferably, 70-100% amino acid identity (i.e., 90, 91, 92, 93, 94, 95, 96, 97, 98, 99, 100 or any range or value therein) is determined using a suitable computer algorithm, as known in the art.

“Identity,” as known in the art, is a relationship between two or more polypeptide sequences or two or more polynucleotide sequences, as determined by comparing the sequences. In the art, “identity” also means the degree of sequence relatedness between polypeptide or polynucleotide sequences, as determined by the match between strings of such sequences. “Identity” and “similarity” can be readily calculated by known methods, including, but not limited to, those described in Computational Molecular Biology, Lesk, A. M., ed., Oxford University Press, New York, 1988; Biocomputing:Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991; and Carillo, H., and Lipman, D., Siam J. Applied Math., 48:1073 (1988). In addition, values for percentage identity can be obtained from amino acid and nucleotide sequence alignments generated using the default settings for the AlignX component of Vector NTI Suite 8.0 (Informax, Frederick, MD).

Preferred methods to determine identity are designed to give the largest match between the sequences tested. Methods to determine identity and similarity are codified in publicly available computer programs. Preferred computer program methods to determine identity and similarity between two sequences include, but are not limited to, the GCG program package (Devereux, J., et al., Nucleic Acids Research 12(1): 387 (1984)), BLASTP, BLASTN, and FASTA (Atschul, S. F. et al., J. Molec. Biol. 215:403-410 (1990)). The BLAST X program is publicly available from NCBI and other sources (BLAST Manual, Altschul, S., et al., NCBINLM NIH Bethesda, Md. 20894: Altschul, S., et al., J. Mol. Biol. 215:403-410 (1990). The well-known Smith Waterman algorithm may also be used to determine identity.

Preferred parameters for polypeptide sequence comparison include the following:
(1) Algorithm: Needleman and Wunsch, J. Mol Biol. 48:443-453 (1970) Comparison matrix: BLOSSUM62 from Hentikoff and Hentikoff, Proc. Natl. Acad. Sci, USA. 89:10915-10919 (1992)
Gap Penalty: 12
Gap Length Penalty: 4
A program useful with these parameters is publicly available as the “gap” program from Genetics Computer Group, Madison Wis. The aforementioned parameters are the default parameters for peptide sequence comparisons (along with no penalty for end gaps).

Preferred parameters for polynucleotide comparison include the following:
(1) Algorithm: Needleman and Wunsch, J. Mol Biol. 48:443-453 (1970)
Comparison matrix: matches=+10, mismatch=0
Gap Penalty: 50
Gap Length Penalty: 3
Available as: The “gap” program from Genetics Computer Group, Madison Wis. These are the default parameters for nucleic acid sequence comparisons.

By way of example, a polynucleotide sequence may be identical to another sequence, that is 100% identical, or it may include up to a certain integer number of nucleotide alterations as compared to the reference sequence. Such alterations are selected from the group consisting of at least one nucleotide deletion, substitution, including transition and transversion, or insertion, and wherein the alterations may occur at the 5′ or 3′ terminal positions of the reference nucleotide sequence or anywhere between those terminal positions, interspersed either individually among the nucleotides in the reference sequence or in one or more contiguous groups within the reference sequence. The number of nucleotide alterations is determined by multiplying the total number of nucleotides in the sequence by the numerical percent of the respective percent identity (divided by 100) and subtracting that product from the total number of nucleotides in the sequence, or:
n.sub.n.ltorsim.x.sub.n−(x.sub.n.y),
wherein n.sub.n is the number of nucleotide alterations, x.sub.n is the total number of nucleotides in sequence, and y is, for instance, 0.70 for 70%, 0.80 for 80%, 0.85 for 85%, 0.90 for 90%, 0.95 for 95%, etc., and wherein any non-integer product of x.sub.n and y is rounded down to the nearest integer prior to subtracting from x.sub.n.

Alterations of a polynucleotide sequence encoding the SEQ ID NOs above may create nonsense, missense or frameshift mutations in this coding sequence and thereby alter the polypeptide encoded by the polynucleotide following such alterations. Similarly, a polypeptide sequence may be identical to the reference sequence of the SEQ ID NOs above, that is be 100% identical, or it may include up to a certain integer number of amino acid alterations as compared to the reference sequence such that the percentage identity is less than 100%. Such alterations are selected from the group consisting of at least one amino acid deletion, substitution, including conservative and non-conservative substitution, or insertion, and wherein the alterations may occur at the amino- or carboxy-terminal positions of the reference polypeptide sequence or anywhere between those terminal positions, interspersed either individually among the amino acids in the reference sequence or in one or more contiguous groups within the reference sequence. The number of amino acid alterations for a given % identity is determined by multiplying the total number of amino acids in the SEQ ID NOs above by the numerical percent of the respective percent identity (divided by 100) and then subtracting that product from the total number of amino acids in the SEQ ID NOs above, or:
n.sub.a.ltorsim.x.sub.a−(x.sub.a.y),
wherein n.sub.a is the number of amino acid alterations, x.sub.a is the total number of amino acids in the SEQ ID NOs above, and y is, for instance 0.70 for 70%, 0.80 for 80%, 0.85 for 85% etc., and wherein any non-integer produce of x.sub.a and y is rounded down to the nearest integer prior to subtracting it from x.sub.a.

Exemplary heavy chain and light chain variable regions sequences and portions thereof are provided in the SEQ ID NOs above. The antibodies of the present invention, or specified variants thereof, can comprise any number of contiguous amino acid residues from an antibody of the present invention, wherein that number is selected from the group of integers consisting of from 10-100% of the number of contiguous residues in an anti-IL-23 antibody.

The method of the present invention also uses an anti-IL-23 antibody composition comprising at least one, at least two, at least three, at least four, at least five, at least six or more anti-IL-23 antibodies thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the anti-IL-23 antibody amino acid sequence selected from the group consisting of 70-100% of the contiguous amino acids of the SEQ ID NOs above, or specified fragments, domains or variants thereof. Preferred anti-IL-23 antibody compositions include at least one or two full length, fragments, domains or variants as at least one CDR or LBP containing portions of the anti-IL-23 antibody sequence described herein, for example, 70-100% of the SEQ ID NOs above, or specified fragments, domains or variants thereof. Further preferred compositions comprise, for example, 40-99% of at least one of 70-100% of the SEQ ID NOs above, etc., or specified fragments, domains or variants thereof. Such composition percentages are by weight, volume, concentration, molarity, or molality as liquid or dry solutions, mixtures, suspension, emulsions, particles, powder, or colloids, as known in the art or as described herein.

Antibody Compositions Comprising Further Therapeutically Active Ingredients

The antibody compositions used in the method of the invention can optionally further comprise an effective amount of at least one compound or protein selected from at least one of an anti-infective drug, a cardiovascular (CV) system drug, a central nervous system (CNS) drug, an autonomic nervous system (ANS) drug, a respiratory tract drug, a gastrointestinal (GI) tract drug, a hormonal drug, a drug for fluid or electrolyte balance, a hematologic drug, an antineoplastic, an immunomodulation drug, an ophthalmic, otic or nasal drug, a topical drug, a nutritional drug or the like. Such drugs are well known in the art, including formulations, indications, dosing and administration for each presented herein (see, e.g., Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, NJ; Pharmcotherapy Handbook, Wells et al., ed., Appleton & Lange, Stamford, CT, each entirely incorporated herein by reference).

By way of example of the drugs that can be combined with the antibodies for the method of the present invention, the anti-infective drug can be at least one selected from amebicides or at least one antiprotozoals, anthelmintics, antifungals, antimalarials, antituberculotics or at least one antileprotics, aminoglycosides, penicillins, cephalosporins, tetracyclines, sulfonamides, fluoroquinolones, antivirals, macrolide anti-infectives, and miscellaneous anti-infectives. The hormonal drug can be at least one selected from corticosteroids, androgens or at least one anabolic steroid, estrogen or at least one progestin, gonadotropin, antidiabetic drug or at least one glucagon, thyroid hormone, thyroid hormone antagonist, pituitary hormone, and parathyroid-like drug. The at least one cephalosporin can be at least one selected from cefaclor, cefadroxil, cefazolin sodium, cefdinir, cefepime hydrochloride, cefixime, cefmetazole sodium, cefonicid sodium, cefoperazone sodium, cefotaxime sodium, cefotetan disodium, cefoxitin sodium, cefpodoxime proxetil, cefprozil, ceftazidime, ceftibuten, ceftizoxime sodium, ceftriaxone sodium, cefuroxime axetil, cefuroxime sodium, cephalexin hydrochloride, cephalexin monohydrate, cephradine, and loracarbef.

The at least one corticosteroid can be at least one selected from betamethasone, betamethasone acetate or betamethasone sodium phosphate, betamethasone sodium phosphate, cortisone acetate, dexamethasone, dexamethasone acetate, dexamethasone sodium phosphate, fludrocortisone acetate, hydrocortisone, hydrocortisone acetate, hydrocortisone cypionate, hydrocortisone sodium phosphate, hydrocortisone sodium succinate, methylprednisolone, methylprednisolone acetate, methylprednisolone sodium succinate, prednisolone, prednisolone acetate, prednisolone sodium phosphate, prednisolone tebutate, prednisone, triamcinolone, triamcinolone acetonide, and triamcinolone diacetate. The at least one androgen or anabolic steroid can be at least one selected from danazol, fluoxymesterone, methyltestosterone, nandrolone decanoate, nandrolone phenpropionate, testosterone, testosterone cypionate, testosterone enanthate, testosterone propionate, and testosterone transdermal system.

The at least one immunosuppressant can be at least one selected from azathioprine, basiliximab, cyclosporine, daclizumab, lymphocyte immune globulin, muromonab-CD3, mycophenolate mofetil, mycophenolate mofetil hydrochloride, sirolimus, and tacrolimus.

The at least one local anti-infective can be at least one selected from acyclovir, amphotericin B, azelaic acid cream, bacitracin, butoconazole nitrate, clindamycin phosphate, clotrimazole, econazole nitrate, erythromycin, gentamicin sulfate, ketoconazole, mafenide acetate, metronidazole (topical), miconazole nitrate, mupirocin, naftifine hydrochloride, neomycin sulfate, nitrofurazone, nystatin, silver sulfadiazine, terbinafine hydrochloride, terconazole, tetracycline hydrochloride, tioconazole, and tolnaftate. The at least one scabicide or pediculicide can be at least one selected from crotamiton, lindane, permethrin, and pyrethrins. The at least one topical corticosteroid can be at least one selected from betamethasone dipropionate, betamethasone valerate, clobetasol propionate, desonide, desoximetasone, dexamethasone, dexamethasone sodium phosphate, diflorasone diacetate, fluocinolone acetonide, fluocinonide, flurandrenolide, fluticasone propionate, halcionide, hydrocortisone, hydrocortisone acetate, hydrocortisone butyrate, hydrocorisone valerate, mometasone furoate, and triamcinolone acetonide. (See, e.g., pp. 1098-1136 of Nursing 2001 Drug Handbook.)

Anti-IL-23 antibody compositions can further comprise at least one of any suitable and effective amount of a composition or pharmaceutical composition comprising at least one anti-IL-23 antibody contacted or administered to a cell, tissue, organ, animal or patient in need of such modulation, treatment or therapy, optionally further comprising at least one selected from at least one TNF antagonist (e.g., but not limited to a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept, CDP-571, CDP-870, afelimomab, lenercept, and the like), an antirheumatic (e.g., methotrexate, auranofin, aurothioglucose, azathioprine, etanercept, gold sodium thiomalate, hydroxychloroquine sulfate, leflunomide, sulfasalzine), an immunization, an immunoglobulin, an immunosuppressive (e.g., basiliximab, cyclosporine, daclizumab), a cytokine or a cytokine antagonist. Non-limiting examples of such cytokines include, but are not limited to, any of IL-1 to IL-40 et al. (e.g., IL-1, IL-2, etc.). Suitable dosages are well known in the art. See, e.g., Wells et al., eds., Pharmacotherapy Handbook, 2nd Edition, Appleton and Lange, Stamford, C T (2000); PDR Pharmacopoeia, Tarascon Pocket Pharmacopoeia 2000, Deluxe Edition, Tarascon Publishing, Loma Linda, CA (2000), each of which references are entirely incorporated herein by reference.

Anti-IL-23 antibody compounds, compositions or combinations used in the method of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington's Pharmaceutical Sciences, 18^{th} Edition, Mack Publishing Co. (Easton, PA) 1990. Pharmaceutically acceptable carriers can be routinely selected that are suitable for the mode of administration, solubility and/or stability of the anti-IL-23 antibody, fragment or variant composition as well known in the art or as described herein.

Pharmaceutical excipients and additives useful in the present composition include, but are not limited to, proteins, peptides, amino acids, lipids, and carbohydrates (e.g., sugars, including monosaccharides, di-, tri-, tetra-, and oligosaccharides; derivatized sugars, such as alditols, aldonic acids, esterified sugars and the like; and polysaccharides or sugar polymers), which can be present singly or in combination, comprising alone or in combination 1-99.99% by weight or volume. Exemplary protein excipients include serum albumin, such as human serum albumin (HSA), recombinant human albumin (rHA), gelatin, casein, and the like. Representative amino acid/antibody components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

Anti-IL-23 antibody compositions can also include a buffer or a pH adjusting agent; typically, the buffer is a salt prepared from an organic acid or base. Representative buffers include organic acid salts, such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts, such as citrate.

Additionally, anti-IL-23 antibody compositions can include polymeric excipients/additives, such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates, such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the anti-IL-23 antibody, portion or variant compositions according to the invention are known in the art, e.g., as listed in “Remington: The Science & Practice of Pharmacy,” 19^{th} ed., Williams & Williams, (1995), and in the “Physician's Desk Reference,” 52^{nd} ed., Medical Economics, Montvale, NJ (1998), the disclosures of which are entirely incorporated herein by reference. Preferred carrier or excipient materials are carbohydrates (e.g., saccharides and alditols) and buffers (e.g., citrate) or polymeric agents. An exemplary carrier molecule is the mucopolysaccharide, hyaluronic acid, which may be useful for intraarticular delivery.

Formulations

As noted above, the invention provides for stable formulations, which preferably comprise a phosphate buffer with saline or a chosen salt, as well as preserved solutions and formulations containing a preservative as well as multi-use preserved formulations suitable for pharmaceutical or veterinary use, comprising at least one anti-IL-23 antibody in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, 1.0%), 0.1-3% benzyl alcohol (e.g., 0.5, 0.9, 1.1, 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the method of the invention uses an article of manufacture, comprising packaging material and at least one vial comprising a solution of at least one anti-IL-23 specific antibody with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further uses an article of manufacture, comprising packaging material, a first vial comprising lyophilized anti-IL-23 specific antibody, and a second vial comprising an aqueous diluent of prescribed buffer or preservative, wherein said packaging material comprises a label that instructs a patient to reconstitute the anti-IL-23 specific antibody in the aqueous diluent to form a solution that can be held over a period of twenty-four hours or greater.

The anti-IL-23 specific antibody used in accordance with the present invention can be produced by recombinant means, including from mammalian cell or transgenic preparations, or can be purified from other biological sources, as described herein or as known in the art.

The range of the anti-IL-23 specific antibody includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g., isotonicity agents, buffers, antioxidants, and preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably, the formulations of the present invention have a pH between about 6.8 and about 7.8. Preferred buffers include phosphate buffers, most preferably, sodium phosphate, particularly, phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants, such as polysorbate 20 or 80 or poloxamer 184 or 188, Pluronic® polyls, other block co-polymers, and chelators, such as EDTA and EGTA, can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations can be prepared by a process which comprises mixing at least one anti-IL-23 specific antibody and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one anti-IL-23 specific antibody and preservative in an aqueous diluent is carried out using conventional dissolution and mixing procedures. To prepare a suitable formulation, for example, a measured amount of at least one anti-IL-23 specific antibody in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The formulations can be provided to patients as clear solutions or as dual vials comprising a vial of lyophilized anti-IL-23 specific antibody that is reconstituted with a second vial containing water, a preservative and/or excipients, preferably, a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present articles of manufacture are useful for administration over a period ranging from immediate to twenty-four hours or greater. Accordingly, the presently claimed articles of manufacture offer significant advantages to the patient. Formulations of the invention can optionally be safely stored at temperatures of from about 2° C. to about 40° C. and retain the biologically activity of the protein for extended periods of time, thus allowing a package label indicating that the solution can be held and/or used over a period of 6, 12, 18, 24, 36, 48, 72, or 96 hours or greater. If preserved diluent is used, such label can include use up to 1-12 months, one-half, one and a half, and/or two years.

The solutions of anti-IL-23 specific antibody can be prepared by a process that comprises mixing at least one antibody in an aqueous diluent. Mixing is carried out using conventional dissolution and mixing procedures. To prepare a suitable diluent, for example, a measured amount of at least one antibody in water or buffer is combined in quantities sufficient to provide the protein and, optionally, a preservative or buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed products can be provided to patients as clear solutions or as dual vials comprising a vial of lyophilized at least one anti-IL-23 specific antibody that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one anti-IL-23 specific antibody that is reconstituted with a second vial containing the aqueous diluent. The clear solution in this case can be up to one liter or even larger in size, providing a large reservoir from which smaller portions of the at least one antibody solution can be retrieved one or multiple times for transfer into smaller vials and provided by the pharmacy or clinic to their customers and/or patients.

Recognized devices comprising single vial systems include pen-injector devices for delivery of a solution, such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, Iject®, J-tip Needle-Free Injector®, Intraject®, Medi-Ject®, Smartject® e.g., as made or developed by Becton Dickensen (Franklin Lakes, NJ, www.bectondickenson.com), Disetronic (Burgdorf, Switzerland, www.disetronic.com; Bioject, Portland, Oregon (www.bioject.com); National Medical Products, Weston Medical (Peterborough, UK, www.weston-medical.com), Medi-Ject Corp (Minneapolis, MN, www.mediject.com), and similarly suitable devices. Recognized devices comprising a dual vial system include those pen-injector systems for reconstituting a lyophilized drug in a cartridge for delivery of the reconstituted solution, such as the HumatroPen®. Examples of other devices suitable include pre-filled syringes, auto-injectors, needle free injectors, and needle free IV infusion sets.

The products may include packaging material. The packaging material provides, in addition to the information required by the regulatory agencies, the conditions under which the product can be used. The packaging material of the present invention provides instructions to the patient, as applicable, to reconstitute the at least one anti-IL-23 antibody in the aqueous diluent to form a solution and to use the solution over a period of 2-24 hours or greater for the two vial, wet/dry, product. For the single vial, solution product, pre-filled syringe or auto-injector, the label indicates that such solution can be used over a period of 2-24 hours or greater. The products are useful for human pharmaceutical product use.

The formulations used in the method of the present invention can be prepared by a process that comprises mixing an anti-IL-23 antibody and a selected buffer, preferably, a phosphate buffer containing saline or a chosen salt. Mixing the anti-IL-23 antibody and buffer in an aqueous diluent is carried out using conventional dissolution and mixing procedures. To prepare a suitable formulation, for example, a measured amount of at least one antibody in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The method of the invention provides pharmaceutical compositions comprising various formulations useful and acceptable for administration to a human or animal patient. Such pharmaceutical compositions are prepared using water at “standard state” as the diluent and routine methods well known to those of ordinary skill in the art. For example, buffering components such as histidine and histidine monohydrochloride hydrate, may be provided first followed by the addition of an appropriate, non-final volume of water diluent, sucrose and polysorbate 80 at “standard state.” Isolated antibody may then be added. Last, the volume of the pharmaceutical composition is adjusted to the desired final volume under “standard state” conditions using water as the diluent. Those skilled in the art will recognize a number of other methods suitable for the preparation of the pharmaceutical compositions.

The pharmaceutical compositions may be aqueous solutions or suspensions comprising the indicated mass of each constituent per unit of water volume or having an indicated pH at “standard state.” As used herein, the term “standard state” means a temperature of 25° C.+/−2° C. and a pressure of 1 atmosphere. The term “standard state” is not used in the art to refer to a single art recognized set of temperatures or pressure, but is instead a reference state that specifies temperatures and pressure to be used to describe a solution or suspension with a particular composition under the reference “standard state” conditions. This is because the volume of a solution is, in part, a function of temperature and pressure. Those skilled in the art will recognize that pharmaceutical compositions equivalent to those disclosed here can be produced at other temperatures and pressures. Whether such pharmaceutical compositions are equivalent to those disclosed here should be determined under the “standard state” conditions defined above (e.g. 25° C.+/−2° C. and a pressure of 1 atmosphere).

Importantly, such pharmaceutical compositions may contain component masses “about” a certain value (e.g. “about 0.53 mg L-histidine”) per unit volume of the pharmaceutical composition or have pH values about a certain value. A component mass present in a pharmaceutical composition or pH value is “about” a given numerical value if the isolated antibody present in the pharmaceutical composition is able to bind a peptide chain while the isolated antibody is present in the pharmaceutical composition or after the isolated antibody has been removed from the pharmaceutical composition (e.g., by dilution). Stated differently, a value, such as a component mass value or pH value, is “about” a given numerical value when the binding activity of the isolated antibody is maintained and detectable after placing the isolated antibody in the pharmaceutical composition.

Competition binding analysis is performed to determine if the IL-23 specific mAbs bind to similar or different epitopes and/or compete with each other. Abs are individually coated on ELISA plates. Competing mAbs are added, followed by the addition of biotinylated hrIL-23. For positive control, the same mAb for coating may be used as the competing mAb (“self-competition”). IL-23 binding is detected using streptavidin. These results demonstrate whether the mAbs recognize similar or partially overlapping epitopes on IL-23.

One aspect of the method of the invention administers to a patient a pharmaceutical composition comprising

In one embodiment of the pharmaceutical compositions, the isolated antibody concentration is from about 77 to about 104 mg per ml of the pharmaceutical composition. In another embodiment of the pharmaceutical compositions the pH is from about 5.5 to about 6.5.

The stable or preserved formulations can be provided to patients as clear solutions or as dual vials comprising a vial of lyophilized at least one anti-IL-23 antibody that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

Other formulations or methods of stabilizing the anti-IL-23 antibody may result in other than a clear solution of lyophilized powder comprising the antibody. Among non-clear solutions are formulations comprising particulate suspensions, said particulates being a composition containing the anti-IL-23 antibody in a structure of variable dimension and known variously as a microsphere, microparticle, nanoparticle, nanosphere, or liposome. Such relatively homogenous, essentially spherical, particulate formulations containing an active agent can be formed by contacting an aqueous phase containing the active agent and a polymer and a nonaqueous phase followed by evaporation of the nonaqueous phase to cause the coalescence of particles from the aqueous phase as taught in U.S. Pat. No. 4,589,330. Porous microparticles can be prepared using a first phase containing active agent and a polymer dispersed in a continuous solvent and removing said solvent from the suspension by freeze-drying or dilution-extraction-precipitation as taught in U.S. Pat. No. 4,818,542. Preferred polymers for such preparations are natural or synthetic copolymers or polymers selected from the group consisting of gleatin agar, starch, arabinogalactan, albumin, collagen, polyglycolic acid, polylactic aced, glycolide-L(−) lactide poly(episilon-caprolactone, poly(epsilon-caprolactone-CO-lactic acid), poly(epsilon-caprolactone-CO-glycolic acid), poly(ß-hydroxy butyric acid), polyethylene oxide, polyethylene, poly(alkyl-2-cyanoacrylate), poly(hydroxyethyl methacrylate), polyamides, poly(amino acids), poly(2-hydroxyethyl DL-aspartamide), poly(ester urea), poly(L-phenylalanine/ethylene glycol/1,6-diisocyanatohexane) and poly(methyl methacrylate). Particularly preferred polymers are polyesters, such as polyglycolic acid, polylactic aced, glycolide-L(−) lactide poly(episilon-caprolactone, poly(epsilon-caprolactone-CO-lactic acid), and poly(epsilon-caprolactone-CO-glycolic acid. Solvents useful for dissolving the polymer and/or the active include: water, hexafluoroisopropanol, methylenechloride, tetrahydrofuran, hexane, benzene, or hexafluoroacetone sesquihydrate. The process of dispersing the active containing phase with a second phase may include pressure forcing said first phase through an orifice in a nozzle to affect droplet formation.

Dry powder formulations may result from processes other than lyophilization, such as by spray drying or solvent extraction by evaporation or by precipitation of a crystalline composition followed by one or more steps to remove aqueous or nonaqueous solvent. Preparation of a spray-dried antibody preparation is taught in U.S. Pat. No. 6,019,968. The antibody-based dry powder compositions may be produced by spray drying solutions or slurries of the antibody and, optionally, excipients, in a solvent under conditions to provide a respirable dry powder. Solvents may include polar compounds, such as water and ethanol, which may be readily dried. Antibody stability may be enhanced by performing the spray drying procedures in the absence of oxygen, such as under a nitrogen blanket or by using nitrogen as the drying gas. Another relatively dry formulation is a dispersion of a plurality of perforated microstructures dispersed in a suspension medium that typically comprises a hydrofluoroalkane propellant as taught in WO 9916419. The stabilized dispersions may be administered to the lung of a patient using a metered dose inhaler. Equipment useful in the commercial manufacture of spray dried medicaments are manufactured by Buchi Ltd. or Niro Corp.

An anti-IL-23 antibody in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating mild to moderate psoriasis, in a cell, tissue, organ, animal, or patient, as known in the art or as described herein, using at least one IL-23 antibody of the present invention, e.g., administering or contacting the cell, tissue, organ, animal, or patient with a therapeutic effective amount of IL-23 specific antibody.

Any method of the present invention can comprise administering an effective amount of a composition or pharmaceutical composition comprising an anti-IL-23 antibody to a cell, tissue, organ, animal or patient in need of such modulation, treatment or therapy. Such a method can optionally further comprise co-administration or combination therapy for treating such diseases or disorders, wherein the administering of said at least one anti-IL-23 antibody, specified portion or variant thereof, further comprises administering, before concurrently, and/or after, at least one selected from at least one TNF antagonist (e.g., but not limited to, a TNF chemical or protein antagonist, TNF monoclonal or polyclonal antibody or fragment, a soluble TNF receptor (e.g., p55, p70 or p85) or fragment, fusion polypeptides thereof, or a small molecule TNF antagonist, e.g., TNF binding protein I or II (TBP-1 or TBP-II), nerelimonmab, infliximab, eternacept (Enbrel™), adalimumab (Humira™), CDP-571, CDP-870, afelimomab, lenercept, and the like), an antirheumatic (e.g., methotrexate, auranofin, aurothioglucose, azathioprine, gold sodium thiomalate, hydroxychloroquine sulfate, leflunomide, sulfasalzine), a muscle relaxant, a narcotic, a non-steroid anti-inflammatory drug (NSAID), an analgesic, an anesthetic, a sedative, a local anesthetic, a neuromuscular blocker, an antimicrobial (e.g., aminoglycoside, an antifungal, an antiparasitic, an antiviral, a carbapenem, cephalosporin, a fluoroquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonist. Suitable dosages are well known in the art. See, e.g., Wells et al., eds., Pharmacotherapy Handbook, 2^{nd} Edition, Appleton and Lange, Stamford, C T (2000); PDR Pharmacopoeia, Tarascon Pocket Pharmacopoeia 2000, Deluxe Edition, Tarascon Publishing, Loma Linda, C A (2000); Nursing 2001 Handbook of Drugs, 21^{st} edition, Springhouse Corp., Springhouse, P A, 2001; Health Professional's Drug Guide 2001, ed., Shannon, Wilson, Stang, Prentice-Hall, Inc, Upper Saddle River, NJ, each of which references are entirely incorporated herein by reference.

Therapeutic Treatments

Typically, treatment of mild to moderate psoriasis is affected by administering an effective amount or dosage of an anti-IL-23 antibody composition that total, on average, a range from at least about 0.01 to 500 milligrams of an anti-IL-23 antibody per kilogram of patient per dose, and, preferably, from at least about 0.1 to 100 milligrams antibody/kilogram of patient per single or multiple administration, depending upon the specific activity of the active agent contained in the composition. Alternatively, the effective serum concentration can comprise 0.1-5000 μg/ml serum concentration per single or multiple administrations. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100-500 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19, 19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300, 400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and, preferably, 0.1 to 10 milligrams per kilogram per administration or in sustained release form is effective to obtain desired results.

As a non-limiting example, treatment of humans or animals can be provided as a one-time or periodic dosage of at least one antibody of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or, alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or, alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.001 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the antibody can be formulated as a solution, suspension, emulsion, particle, powder, or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles, such as fixed oils, can also be used. The vehicle or lyophilized powder can contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Alternative Administration

Many known and developed modes can be used according to the present invention for administering pharmaceutically effective amounts of an anti-IL-23 antibody. While pulmonary administration is used in the following description, other modes of administration can be used according to the present invention with suitable results. IL-23 specific antibodies of the present invention can be delivered in a carrier, as a solution, emulsion, colloid, or suspension, or as a dry powder, using any of a variety of devices and methods suitable for administration by inhalation or other modes described here within or known in the art.

Parenteral Formulations and Administration

Formulations for parenteral administration can contain as common excipients sterile water or saline, polyalkylene glycols, such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent, such as aqueous solution, a sterile injectable solution or suspension in a solvent. As the usable vehicle or solvent, water, Ringer's solution, isotonic saline, etc. are allowed; as an ordinary solvent or suspending solvent, sterile involatile oil can be used. For these purposes, any kind of involatile oil and fatty acid can be used, including natural or synthetic or semisynthetic fatty oils or fatty acids; natural or synthetic or semisynthetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of an anti-IL-23 antibody by parenteral, subcutaneous, intramuscular, intravenous, intrarticular, intrabronchial, intraabdominal, intracapsular, intracartilaginous, intracavitary, intracelial, intracelebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, intralesional, bolus, vaginal, rectal, buccal, sublingual, intranasal, or transdermal means. An anti-IL-23 antibody composition can be prepared for use for parenteral (subcutaneous, intramuscular or intravenous) or any other administration particularly in the form of liquid solutions or suspensions; for use in vaginal or rectal administration particularly in semisolid forms, such as, but not limited to, creams and suppositories; for buccal, or sublingual administration, such as, but not limited to, in the form of tablets or capsules; or intranasally, such as, but not limited to, the form of powders, nasal drops or aerosols or certain agents; or transdermally, such as not limited to a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement;” Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways, such as electroporation, or to increase the mobility of charged drugs through the skin, such as iontophoresis, or application of ultrasound, such as sonophoresis (U.S. Pat. Nos. 4,309,989 and 4,767,402) (the above publications and patents being entirely incorporated herein by reference).

Having generally described the invention, the same will be more readily understood by reference to the following Examples, which are provided by way of illustration and are not intended as limiting. Further details of the invention are illustrated by the following non-limiting Examples. The disclosures of all citations in the specification are expressly incorporated herein by reference.

### Example 1—Treatment of Mild to Moderate Psoriasis with IL23 Antibody

A Phase 3, Multicenter, Randomized, Double-blind, Placebo- and Active Comparator-Controlled Study Evaluating the Safety and Efficacy of Guselkumab versus Placebo and Apremilast for the Treatment of Mild to Moderate Psoriasis (SPECTREM)

The primary objective of this study is to evaluate the efficacy of guselkumab versus placebo and apremilast in participants with mild to moderate psoriasis. Other secondary objectives of this study are safety, tolerability, pharmacokinetics, pharmacodynamics, and immunogenicity.

This study will directly investigate the safety and efficacy of guselkumab 100 mg subcutaneous (SC) injection given at Weeks 0, 4 and then every 8 weeks (q8w) compared with both placebo and apremilast 30 mg twice daily (BID) after standard up-titration, in the treatment of participants with mild to moderate plaque psoriasis.

The primary endpoint will be the proportion of participants with an Investigator Global Assessment (IGA) score of 0 (cleared) or 1 (minimal) in those with at least a 2-point reduction from baseline at Week 16 compared to placebo. The first major secondary endpoint will be IGA 0/1 at Week 16 compared to the active comparator (apremilast). Additional controlled secondary endpoints and other endpoints are outlined in the endpoints section below.

Approximately 450 eligible participants will be randomized 1:1:1 to receive either guselkumab 100 mg SC at Weeks 0 and 4 and then q8w, apremilast standard uptitration followed by 30 mg twice a day (BID), or placebo. Randomization will be stratified by baseline IGA score (mild [2] or moderate [3]) and study site to ensure balance between treatment arms with respect to baseline severity of psoriasis. Approximately 30% of participants randomized will have a baseline IGA score of mild (2) and approximately 70% of participants will have a baseline IGA score of moderate (3).

There will be 2 database locks (DBLs) in this study, at Week 24 and Week 56. After all participants have completed the Week 24 Visit (or discontinued from the study), a Week 24 DBL will be performed, and unblinded data will only be made available to select Sponsor and Contract Research Organization (CRO) team members involved with analysis of the data and preparation of analyses for the Week 24 DBL. All other Sponsor, site, and CRO personnel directly involved with study conduct will remain blinded to treatment assignments until the Week 56 DBL and related analyses have been completed.

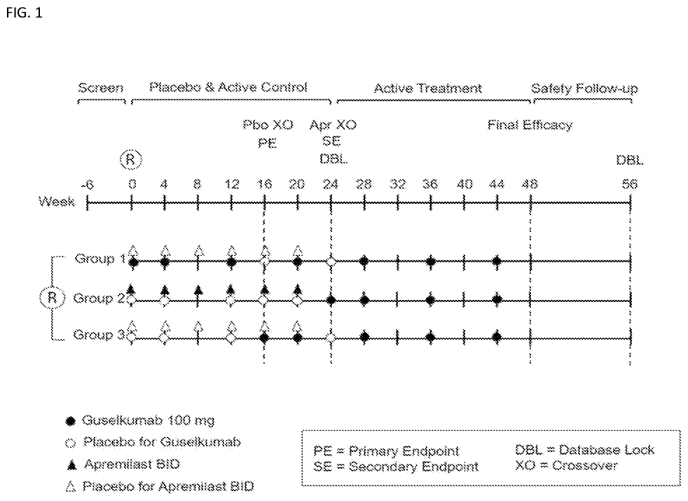

The study design and treatment assignments are further described below and in FIG. 1.

### Study Population

The target population is adult men or women, with a diagnosis of mild to moderate plaque-type psoriasis with or without PsA, for at least 6 months. Participants with non-plaque forms of psoriasis (e.g., erythrodermic, guttate or pustular) or with drug-induced psoriasis (e.g., new onset or exacerbation of psoriasis from beta blockers, calcium channel blockers or lithium) are excluded.

Participants must have mild to moderate psoriasis defined as BSA 2-15%, IGA 2 or 3, and PASI 2-15. Participants must be candidates for either systemic therapy or phototherapy for psoriasis and must have inadequate response or intolerance to at least 1 prior topical therapy. Participants must be naïve to advanced therapies (i.e., no prior exposure for the treatment of psoriasis, psoriatic arthritis, or any other indication that could impact the assessment of psoriasis), including apremilast, guselkumab, and other oral immunomodulatory therapies or biologic therapies.

### Inclusion Criteria—Participant Population

1 Participant sex (biologically): Both males and females

### 2 Participant age:

3 Minimum age: 18 (or the legal age of consent in the jurisdiction in which the study is taking place)
4 Have a diagnosis of plaque psoriasis (with or without PsA) for at least 6 months before the first administration of study drug
5 Be a candidate for phototherapy or systemic treatment for psoriasis
6 Have an involved BSA 2%-15% at screening and at baseline
7 Have an IGA 2 or 3 at screening and at baseline
8 Have a PASI 2-15 at screening and at baseline
9 Participant must be inadequately controlled with, or intolerant of at least one topical therapy (including topical corticosteroids, topical retinoids, or vitamin D analog preparations, calcipotriene and betamethasone dipropionate ointment or foam, tacrolimus, pimecrolimus, or anthralin/dithranol) for the treatment of psoriasis at both screening and baseline
10 Be naïve to advanced therapies (i.e., no prior exposure for the treatment of psoriasis, psoriatic arthritis, or any other indication that could impact the assessment of psoriasis), including apremilast, guselkumab, and other oral immunomodulatory therapies or biologic therapies
11 Be considered, in the opinion of the investigator, suitable candidates for apremilast therapy according to their country's approved Otezla® product labeling
12 Before the first administration of study drug, a woman must be either:
a. Not of childbearing potential: premenarchal; postmenopausal (>45 years of age with amenorrhea for at least 12 months or any age with amenorrhea for at least 6 months and a serum follicle-stimulating hormone level >40 IU/L); permanently sterilized (e.g., tubal occlusion/ligation, hysterectomy, bilateral salpingectomy); or otherwise, incapable of pregnancy
b. Of childbearing potential and practicing a highly effective method of birth control, consistent with local regulations regarding the use of birth control methods for participants participating in clinical studies: e.g., established use of oral, injected, or implanted hormonal methods of contraception; placement of an intrauterine device or intrauterine system; barrier methods: condom or occlusive cap (diaphragm or cervical/vault caps) plus spermicidal foam/gel/film/cream/suppository; male partner sterilization (the vasectomized partner should be the sole partner for that participant); true abstinence (when this is in line with the preferred and usual lifestyle of the participant)
Note: If a female participant's childbearing potential changes after the start of the study (eg, a woman who is not heterosexually active becomes active, a premenarchal woman experiences menarche), she must begin practicing a highly effective method of birth control, as described
13 A woman of childbearing potential must have a negative urine pregnancy test at screening at Week 0 and agree to urine pregnancy testing before receiving injections
14 A woman must agree not to donate eggs (ova, oocytes) for the purposes of assisted reproduction during the study and for at least 12 weeks after receiving the last administration of guselkumab
15 A man who is sexually active with a woman of childbearing potential and who has not had a vasectomy must agree to use a barrier method of birth control (e.g., either a condom [with spermicidal foam/gel/film/cream/suppository] or a partner with an occlusive cap [diaphragm or cervical/vault caps] plus spermicidal foam/gel/film/cream/suppository), during the study and for at least 12 weeks after receiving the last administration of guselkumab. All men must also agree to not donate sperm during the study and for at least 12 weeks after receiving the last administration of guselkumab.

### Infectious Disease-Related

16 Agree not to receive a live virus or live bacterial vaccination during the study, or within
12 weeks after the last administration of study intervention.
17 Agree not to receive a Bacillus Calmette-Guérin (BCG) vaccination during the study, and within
12 weeks after the last administration of study intervention.
18 It is recommended that patients are up-to-date on age-appropriate vaccinations prior to screening
as per routine local medical guidelines. For study patients who received locally approved (and including emergency use-authorized) COVID-19 vaccines recently prior to study entry, follow applicable local vaccine labelling, guidelines, and standards of care for patients receiving immune-targeted therapy when determining an appropriate interval between vaccination and study enrolment.
19 Have screening laboratory test results within the following parameters, if one or more of the laboratory parameters is out of range, a single retest of laboratory values is permitted:
a. Hemoglobin ≥10 g/dL (SI: ≥100 g/L)
b. White blood cells ≥3.5×103/μL (SI: ≥3.5 GI/L)
c. Neutrophils ≥1.5×103/μL (SI: ≥1.5 GI/L)
d. Platelets ≥100×103/μL (SI: ≥100 GI/L)
e. Serum creatinine ≤1.5 mg/dL (SI: ≤137 μmol/L)
f. Aspartate aminotransferase ≤2×upper limit of normal (ULN)
g. Alanine aminotransferase ≤2×ULN
NOTE: A 1-time repeat of screening laboratory tests (a-e) is allowed during the 6-week screening phase and the investigator may consider the participant eligible if the previously abnormal laboratory test result is within an acceptable range on repeat testing in the central laboratory.

### Other

20 Agree to avoid prolonged sun exposure and avoid use of tanning booths or other ultraviolet light
sources during study.
21 Be willing and able to adhere to the prohibitions and restrictions specified in this protocol.
22 Must sign an informed consent form (ICF) [(or their legally acceptable representative must sign)] indicating that he or she understands the purpose of, and procedures required for, the study and is willing to participate in the study.

### Exclusion Criteria—Psoriasis

1 Has a non-plaque form of psoriasis (e.g., erythrodermic, guttate, or pustular).
2 Has current drug-induced psoriasis (e.g., a new onset of psoriasis or an exacerbation of psoriasis
from beta blockers, calcium channel blockers, or lithium).
3 Has psoriasis flare/rebound (defined as a sudden worsening of psoriasis which requires administration of prohibited medications) within 4 weeks of signing the ICF or between the Screening and Baseline Visit.
4 Has previously received any biologics used to treat psoriasis, psoriatic psoriasis, or any other indications that could impact the assessment of psoriasis including but not limited to
TNFinhibitors (e.g., adalimumab, etanercept, infliximab, or certolizumab), IL-17 inhibitors
(secukinumab, ixekizumab), IL-12/23 inhibitor (ustekinumab), IL-23 inhibitor (e.g., guselkumab, risankizumab, or tildrakizumab, etc.).
5 Has previously received apremilast.
6 Has received phototherapy within 4 weeks of the first administration of study intervention.
7 Has received any systemic immunosuppressants (eg, methotrexate [MTX], azathioprine, cyclosporine, 6-thioguanine, mercaptopurine, mycophenolate mofetil, tacrolimus, acitretin) or anakinra within 4 weeks of the first administration of study drug.
8 Has received any systemic medications that could affect psoriasis or IGA evaluations including but not limited to oral or injectable corticosteroids, retinoids, 1, 25 dihydroxy vitamin D3 and analogues, psoralens, sulfasalazine, hydroxyurea, fumaric acid derivatives within 4 weeks of the first administration of study drug.
9 Has used topical medications that could affect psoriasis or IGA evaluations (including, but not limited to topical corticosteroids, topical retinoids, or Vitamin D analog preparations, calcipotriene and betamethasone dipropionate ointment or foam, tacrolimus, pimecrolimus, or anthralin/dithranol, experimental topicals like taparinof or roflumilast, coal tar derivatives within 2 weeks of the first administration of study drug.
10 Has received herbal treatments, or traditional Taiwanese, Korean, or Chinese medicines that could affect psoriasis or IGA evaluations within 4 weeks of the first administration of study drug.
11 Has received a biologic therapy or an experimental antibody or within the previous 12 weeks or
5 half-lives (whichever is longer) of any study drug administration or is currently enrolled in another study using an investigational agent or procedure.
12 Is currently receiving lithium, antimalarials, or intramuscular (IM) gold, or have received lithium, antimalarials, or IM gold within 4 weeks of the first administration of study drug.

### Coexisting Medical Conditions or Past History

13 Has a history or current signs or symptoms of severe, progressive, or uncontrolled renal, cardiac,
vascular, pulmonary, gastrointestinal, endocrine, neurologic, hematologic, rheumatologic, psychiatric, or metabolic disturbances.
14 Has unstable cardiovascular disease, defined as a recent clinical deterioration (eg, unstable angina, rapid atrial fibrillation) in the last 3 months or a cardiac hospitalization within the last 3 months.
15 Currently has a known malignancy or has a history of malignancy within 5 years before screening (with the exception of a nonmelanoma skin cancer that has been adequately treated with no evidence of recurrence for at least 3 months before the first study drug administration or cervical carcinoma in situ that has been treated with no evidence of recurrence for at least 3 months before the first study drug administration).
16 Has a history of lymphoproliferative disease, including lymphoma; a history of monoclonal gammopathy of undetermined significance; or signs and symptoms suggestive of possible lymphoproliferative disease, such as lymphadenopathy or splenomegaly.
17 Has a transplanted organ (with exception of a corneal transplant >3 months before the first administration of study drug).
18 Has known intolerance or hypersensitivity to any biologic medication, or known allergies or clinically significant reactions to murine, chimeric, or human proteins, mAbs, or antibody fragments.
19 Has prior history of suicide attempt at any time in the participant's life-time prior to signing the
informed consent and randomization, or major psychiatric illness requiring hospitalization within the last 3 years prior to signing the informed consent.
20 Has unstable suicidal ideation or suicidal behavior, that may be defined as an electronic Columbia-Suicide Severity Rating Scale (eC-SSRS) rating at screening of: Suicidal ideation with intention to act (“4”), Suicidal ideation with specific plan and intent (“5”), or a suicide attempt (interrupted suicide attempt, aborted suicide attempt, or preparatory behaviors for making a suicide attempt) in the last 6 months and is confirmed to be at risk by the investigator based on an evaluation by a mental health professional. The final decision on excluding a participant will be made at the judgment of a trained mental health professional.
21 Has a history of an infected joint prosthesis or has received antibiotics for a suspected infection of a joint prosthesis, if that prosthesis has not been removed or replaced.
22 Has known allergies or hypersensitivity to any components of apremilast.
23 Has known allergies, hypersensitivity, or intolerance to guselkumab or its excipients (refer to Investigator's Brochure).
24 Is pregnant, nursing, or planning a pregnancy (both men and women) while enrolled in this study
and within 12 weeks following the last administration of study drug.
25 Has had major surgery (eg, requiring general anesthesia and hospitalization) within 8 weeks before screening, or will not have fully recovered from such surgery, or has such surgery planned during the time the participant is expected to participate in the study (56 weeks).
Note: Participants with planned surgical procedures to be conducted under local anesthesia may participate.
26 Is known to have had a substance abuse (drug or alcohol) problem within the previous 12 months.
27 Has evidence of skin conditions that would interfere with clinical assessments of psoriasis

### Infections or Predisposition to Infection

28 Has a history of chronic or recurrent infectious disease, including but not limited to chronic renal
infection, chronic chest infection (eg, bronchiectasis), recurrent urinary tract infection (recurrent pyelonephritis or chronic non-remitting cystitis), fungal infection (mucocutaneous candidiasis), or open, draining, or infected skin wounds or ulcers.
29 Has or has had a serious infection (eg, sepsis, pneumonia, or pyelonephritis), or has been hospitalized or received intravenous antibiotics for a clinically relevant infection during the 2 months before screening.
30 Has or has had herpes zoster within the 2 months before screening.
31 Has received, or is expected to receive, any live virus or bacterial vaccination within 3 months prior to the first administration of study drug, plans to receive such vaccines during the study or within 4 weeks after the last administration of study intervention. For BCG vaccine, see Exclusion Criterion 32.
32 Has had a BCG vaccination within 12 months of screening.
33 Has a chest radiograph within 3 months before the first administration of study drug that shows
an abnormality suggestive of a malignancy or current active infection, including TB.
34 Meet ANY of the following tuberculosis (TB) screening criteria:
a. Have a history of active TB or show signs or symptoms suggestive of active TB upon medical history and/or physical examination at screening.
b. Have a history of untreated latent TB prior to screening. An exception is made for participants who are currently receiving treatment or will initiate treatment for latent TB prior to first administration of study intervention.
Note: For participants with a history of treated latent TB there must be documentation of appropriate treatment prior to the first administration of study intervention. It is the responsibility of the investigator to verify the adequacy of previous TB treatment and provide appropriate documentation. QuantiFERON-TB® (QFT) testing is not required at screening for participants with a history of treated latent TB or ongoing treatment for latent TB.
c. Have had recent close contact with a person with active TB. An exception is made if such participants are referred to a physician specializing in TB to determine if treatment is warranted or not. This evaluation must be adequately documented and, if treatment is recommended, the participant must be receiving appropriate treatment prior to the first administration of study intervention.
d. Have a positive QFT test result within 2 months prior to the first administration of study intervention. An exception is made for participants who:
have a history of adequately treated latent TB described above.
have a newly identified positive QFT test result in which active TB has been ruled out and for which appropriate treatment for latent TB has been initiated prior to the first administration of study intervention.
have a false-positive QFT test as determined by the following:
   A suspected false-positive initial QFT test must be repeated. If repeat testing is NOT positive, the participant must be referred to a physician specializing in TB to determine if the initial test can be considered a false-positive. This evaluation must be adequately documented prior to the first administration of study intervention. If repeat testing is positive, however, it will be considered a true-positive and the participant is only eligible if active TB has been ruled out and appropriate treatment for latent TB has been initiated as described above.
      Note: Indeterminate/borderline results should be handled as outlined in Protocol Section 8.2.7.
      e. Have a chest radiograph or chest computed tomography within 3 months prior to the first administration of study intervention that shows abnormalities suggestive of active or inactive TB.
      35 Tests positive for hepatitis B virus (HBV) infection (Protocol Attachment 5).
      36 Seropositive for antibodies to hepatitis C virus (HCV), unless they satisfy one of the following conditions:
      a. Have a history of successful treatment (defined as being negative for HCV RNA at least 12 weeks after completing antiviral treatment) and have a negative HCV RNA test result at screening, OR
      b. While seropositive have a negative HCV RNA test result at least 12 weeks prior to screening and a negative HCV RNA test result at screening.
      37 Has a history of active granulomatous infection, including histoplasmosis or coccidioidomycosis,
      before screening. Refer to Exclusion Criterion 34 for information regarding eligibility with a history of latent TB.
      38 Has had a nontuberculous mycobacterial infection or opportunistic infection (eg, cytomegalovirus, pneumocystosis, aspergillosis).
      39 Is infected with human immunodeficiency virus (HIV, positive serology for HIV antibody).
      40 During the 6 weeks prior to baseline, have had ANY of
      a) confirmed severe acute respiratory syndrome coronavirus 2 (SARS-CoV-2)/COVID 19 infection (test positive), OR
      b) suspected SARS-CoV-2 infection (clinical features without documented test results),

### OR

c) close contact with a person with known or suspected SARS-Cov-2 infection.
An exception to this criterion may be granted if a participant has a documented negative result for a validated SARS-CoV-2 test:
Obtained at least 2 weeks after conditions (a), (b), (c) above (timed from resolution of key clinical features if present, eg, fever, cough, dyspnea)

### AND

with absence of ALL conditions (a), (b), (c) above during the period between the negative test result and the baseline study visit.
NOTES on COVID-related exclusion:
The field of COVID-related testing (for presence of, and immunity to, the SARS-CoV-2 virus) is rapidly evolving. Additional testing may be performed as part of screening and/or during the study if deemed necessary by the investigator and in accordance with current regulations/guidance from authorities/standards of care.
For those who may carry a higher risk for severe COVID-19 illness, follow guidance from local health authorities when weighing the potential benefits and risks of enrolling in the study, and during participation in the study.

### General

41 Is unable or unwilling to undergo multiple venipunctures because of poor tolerability or lack of
easy access to veins.
42 Lives in an institution on court or authority order.
43 Has any condition that, in the opinion of the investigator, would make participation not be in the
best interest (eg, compromise the well-being) of the participant or that could prevent, limit, or confound the protocol-specified assessments.
44 Is an employee of the investigator or study site, with direct involvement in the proposed study
or
other studies under the direction of that investigator or study site, as well as family members of the employees or the investigator.

### Lifestyle Considerations

Prohibited and restricted therapies during the study:
Corticosteroids (topical, intralesional, shampoo, oral)
Coal Tar, liquor carbonis detergens (LCD), and any other topicals (calcipotriene, tacrolimus, etc) that can be used to treat psoriasis

Participants must avoid prolonged sun exposure and avoid use of tanning booths or other UV light sources during the study.

Permitted Concomitant medications: Topical emollients, eg, Eucerin®, Vaseline®, Lubriderm®,

etc.

### Sequence and Duration of Study Phases/Periods

### 1. Screening Phase—Up to Approximately 6 Weeks

### 2. Blinded Treatment Period—Week 0-24

At Week 0, participants will be randomized to guselkumab (100 mg SC at Weeks 0 and 4 then q8w), apremilast, or placebo in a 1:1:1 ratio per strata for the first 16 weeks
The primary endpoint of IGA 0/1 for guselkumab vs placebo will be evaluated at Week 16
The first major secondary endpoint of IGA 0/1 for guselkumab vs apremilast will be evaluated at Week 16
At Week 16, the participants who had been receiving placebo will switch to guselkumab (Weeks 0 and 4 then q8w) in a blinded fashion; at Week 24, the participants who had been receiving apremilast will switch to guselkumab (Weeks 0 and 4 then q8w) in a blinded fashion
All treatment groups will receive study treatment through Week 44 (last treatment dose)

### 3. Efficacy and Safety Follow-Up Phase—Week 44 to 56

A final efficacy visit will be performed at Week 48
A final safety follow-up visit will occur at Week 56

Choice of Placebo Control and Active Comparator

Placebo

A placebo-controlled design was chosen in order to provide a robust assessment of the efficacy of guselkumab 100 mg SC at Weeks 0, 4, and q8w in mild to moderate psoriasis. The placebo-controlled design is intended to minimize participant and investigator bias in evaluating the efficacy and safety of guselkumab in the selected patient population (Food and Drug Administration [FDA] Guidance for Industry E10).

Otezla (Apremilast)

Apremilast, a specific phosphodiesterase type 4 (PDE4) inhibitor, was approved for moderate to severe plaque psoriasis and active PsA in 2014. In December 2021, it became the first approved advanced oral medication for the treatment of mild to moderate plaque psoriasis given demonstration of statistically significant and clinically meaningful improvements in multiple efficacy measures. Apremilast was selected as the active comparator because as an advanced oral therapy it is safer than conventional systemic therapies like methotrexate or cyclosporine while achieving meaningful levels of efficacy in mild to moderate psoriasis, thereby providing a valuable and relevant benchmark for comparison with guselkumab. Participants randomized to apremilast will be administered doses according to the labeled dose regimen for plaque psoriasis.

Hypothesis

It is hypothesized in the treatment of mild to moderate psoriasis as defined by BSA 2-15%, IGA 2 or 3, and PASI 2-15, (i) guselkumab is superior to placebo and (ii) guselkumab is superior to apremilast. This study is designed to show a difference between guselkumab and placebo at Week 16 and guselkumab and apremilast at both Week 16 and Week 24.

TABLE 1
OBJECTIVES AND ENDPOINTS

| | |
|---|---|
| Primary Objective | Primary Endpoint |
| The primary objective of the | The proportion of participants who achieve an IGA score of cleared |
| study is to evaluate the clinical | (0) or minimal (1) with at least ≥2 grade improvement from |
| efficacy of guselkumab | baseline at Week 16, comparing the guselkumab group and the |
| compared to placebo, in | placebo group |
| participants with mild to | |
| moderate plaque psoriasis | |
| during the 16-week Placebo- | |
| controlled Phase | |
| Secondary Objectives Efficacy | Ranked Major Secondary Efficacy Endpoints |
| To evaluate the efficacy of | 1. The proportion of participants who achieve an IGA score of |
| guselkumab compared with | cleared (0) or minimal (1) with at least ≥2 grade improvement from |
| placebo and apremilast on | baseline at Week 16, comparing the guselkumab group and the |
| improving the signs and | apremilast group |
| symptoms of psoriasis, scalp | 2. The proportion of participants who achieve BSA ≤1% at Week |
| psoriasis, and patient-reported | 16, comparing the guselkumab group and the placebo group |
| Health-related Quality of Life | 3. The proportion of participants who achieve an IGA score of |
| (HRQoL) outcomes | cleared (0) at Week 16, comparing the guselkumab group and the |
| | placebo group |
| | 4. The proportion of participants who achieve a PASI 90 response |
| | at Week 16, comparing the guselkumab group and the placebo |
| | group |
| | 5. The proportion of participants who achieve a PASI 100 response |
| | at Week 16, comparing the guselkumab group and the placebo |
| | group |
| | 6. The proportion of participants who achieve a Scalp-Specific |
| | Investigator's Global Assessment (ss-IGA) score of absence of |
| | disease (0) or very mild disease (1) and have at least a 2-grade |
| | improvement from baseline at Week 16, comparing the |
| | guselkumab group and placebo group, among randomized |
| | participants with scalp psoriasis and an ss-IGA score ≥2 at |
| | baseline |
| | 7. The proportion of participants with ≥4-point reduction |
| | (improvement) in Psoriasis Symptom and Sign Diary (PSSD) |
| | Itch score from baseline at Week 16, comparing the guselkumab |
| | group and placebo group, among participants with a PSSD Itch |
| | score ≥4 at baseline |
| | 8. The change from baseline in PSSD symptom score at Week 16, |
| | comparing the guselkumab group and placebo group |
| | 9. The proportion of participants who achieve BSA ≤1% at |
| | Week 16, comparing the guselkumab group and the apremilast |
| | group |
| | 10. The proportion of participants who achieve an IGA score of |
| | cleared (0) at Week 16, comparing the guselkumab group and |
| | the apremilast group |
| | 11. The proportion of participants who achieve a PASI 90 |
| | response at Week 16, comparing the guselkumab group and the |
| | apremilast group |
| | 12. The proportion of participants who achieve a PASI 100 |
| | response at Week 16, comparing the guselkumab group and the |
| | apremilast group |
| | 13. The proportion of participants who achieve a ss-IGA score of |
| | absence of disease (0) or very mild disease |
| | a. and have at least a 2-grade improvement from baseline at |
| | Week 16, comparing the guselkumab group and apremilast |
| | group, among randomized participants with scalp psoriasis |
| | and an ss-IGA score ≥2 at baseline |
| | 14. The proportion of participants who achieve a PSSD symptom |
| | score of 0 at Week 24 vs apremilast, among randomized |
| | participants with a baseline PSSD symptom score ≥1 |
| | 15. The proportion of participants with ≥4-point reduction |
| | (improvement) in PSSD Itch score from baseline at Week 16, |
| | comparing the guselkumab group and apremilast group, among |
| | participants with a PSSD Itch score ≥4 at baseline |
| | 16. The percent improvement from baseline in the Nail Psoriasis |
| | Severity Index (NAPSI) at Week 16 among randomized |
| | participants with nail psoriasis at baseline |
| Secondary Objectives Safety | Safety Endpoints |
| To evaluate the safety of | The frequency and type of adverse events and serious adverse |
| guselkumab in participants | events |
| with mild to moderate | |
| psoriasis | |
| Other Objectives | Other Prespecified Psoriasis Efficacy Endpoints |
| To evaluate the efficacy of | The change from baseline in BSA % by visit for the guselkumab, |
| guselkumab across additional | placebo, and apremilast groups |
| clinical and patient-reported | The change from baseline in PASI by visit for the guselkumab, |
| HRQoL measures | placebo, and apremilast groups |
| | The proportion of participants who achieve PASI 100 comparing |
| | the guselkumab group and the placebo group over time through |
| | Week 48 |
| | The proportion of participants who achieve BSA ≤1% comparing |
| | the guselkumab group and the placebo group over time through |
| | Week 48 |
| | The proportion of participants who achieve BSA ≤1% comparing |
| | the guselkumab group and the apremilast group over time |
| | through Week 48 |
| | The proportion of participants who achieve PASI 90 comparing |
| | the guselkumab group and the apremilast group over time |
| | through Week 48 |
| | The proportion of participants who achieve PASI 100 comparing |
| | the guselkumab group and the apremilast group over time |
| | through Week 48 |
| | Time to PASI 90 and to PASI 100 for guselkumab vs apremilast |
| | Time to BSA ≤3% or BSA75 for guselkumab vs placebo (NPF |
| | treat to target measure) |
| | The proportions of participants achieving 100% improvement, |
| | ≥90%, 75%, or 50% improvement from baseline in PASI disease |
| | component (Induration, Erythema, and Scaling) and region |
| | component (head, trunk, upper extremities, and lower extremities) |
| | will also be summarized at Week 16, Week 24 and Week 48. |
| | The proportions of participants achieving a IGA score of |
| | cleared (0); IGA score of cleared (0) or minimal (1) and the |
| | proportion of participants achieving a IGA score of mild or |
| | better (≤2) overall and by weight (≤90 kg, >90 kg) will be |
| | summarized by treatment group over time through Week 48. |
| | Other Prespecified Regional Psoriasis Efficacy Endpoints |
| | The proportion of participants with ss-IGA score of absence of |
| | disease (0) or very minimal disease (1) with at least a 2-grade |
| | improvement from baseline at Week 24, comparing the |
| | guselkumab and apremilast group among randomized participants |
| | with scalp psoriasis and an ss-IGA score ≥2 at baseline |
| | The percent improvement from baseline in NAPSI score over |
| | time through Week 48 among randomized participants with nail |
| | psoriasis at baseline comparing the guselkumab, placebo, and |
| | apremilast groups |
| | Change in NAPSI score for guselkumab vs apremilast over time |
| | Proportion of patients achieving NAPSI 0 over time through |
| | Week 48 among randomized participants with nail psoriasis at |
| | baseline comparing the guselkumab, placebo, and apremilast |
| | groups |
| | Change in sPGA-G score for guselkumab vs placebo over time |
| | Change in sPGA-G score for guselkumab vs apremilast over time |
| | Proportion of participants achieving sPGA-G 0/1 with ≥2-point |
| | improvement comparing the guselkumab, placebo, and apremilast |
| | groups over time through Week 48 |
| | Change in Physician Global Assessment of hands and feet |
| | (hfPGA) scores for guselkumab vs placebo over time |
| | Change in hfPGA score for guselkumab vs apremilast over time |
| | The proportion of participants who achieve an hfPGA score of |
| | clear (0) or almost clear (1) and have at least a 2-grade |
| | improvement from baseline at Week 24 and 48 among randomized |
| | participants with hand and/or foot psoriasis and an hf-PGA score |
| | ≥2 at baseline |
| | Other Prespecified Patient-Reported HRQOL Outcome Endpoints |
| | The change from baseline in individual scale score of PSSD |
| | components at Week 16 in guselkumab vs placebo and apremilast |
| | vs placebo groups |
| | The proportion of participants who achieve PSSD individual scale |
| | score of 0 at Week 16 among randomized participants with scale |
| | score ≥1 |
| | The proportion of participants who achieve a PSSD symptom |
| | score =0 at Week 16 among randomized participants with PSSD |
| | symptom score ≥1 |
| | In subjects with PSSD Itch score ≥4 at baseline, proportion of |
| | participants with ≥4-point reduction (improvement) from baseline |
| | either Itch PSSD ≥4 vs placebo at Week 16 |
| | In participants with PSSD Itch score ≥4 at baseline, proportion of |
| | participants with ≥4-point reduction (improvement) from baseline |
| | either Itch PSSD ≥4 vs apremilast at Week 24 |
| | The change in baseline in PSSD symptom score at Week 16, |
| | comparing the guselkumab and placebo group |
| | The change in baseline in PSSD symptom score at Week 16 and |
| | Week 24 comparing the guselkumab and apremilast group |
| | Change in PSSD symptom score from baseline vs apremilast over |
| | time |
| | The proportions of participants who achieve a PSSD symptom |
| | score =0, a PSSD sign score =0, and the proportion of participants |
| | who achieve PSSD individual scale score of 0 for participants |
| | with a baseline symptom score, sign score, and each PSSD |
| | individual baseline scale score that is >0. |
| | The proportion of participants with DLQI 0/1 vs placebo at |
| | Week 16 |
| | The proportion of participants with DLQI 0/1 vs apremilast at |
| | Week 16 and 24 |
| | Change from baseline in patient-reported outcomes measurement |
| | information system-29 (PROMIS-29) score by visit over time |
| | Change in Functional Assessment of Chronic Illness Therapy- |
| | Fatigue (FACIT-F) score by visit over time through Week 48 |
| | Proportion of participants who achieve ≥4-point improvement |
| | from baseline in FACIT-F score by visit over time |
| | The change from baseline in psoriatic arthritis impact of disease |
| | score (PsAID12) by visit over time through Week 48 |
| Other Objectives | Other Prespecified Endpoints |
| To evaluate pharmacokinetics | Serum concentrations of guselkumab |
| and immunogenicity of | Antibodies to guselkumab |
| guselkumab | Change from baseline in cellular and molecular biomarkers in |
| To evaluate pharmacodynamic | skin and blood |
| (PD) effects of guselkumab | Genetic factors associated with clinical response and |
| To evaluate pharmacogenomic | pharmagodynamic (PD) effects |
| of guselkumab | |

TABLE 2
Study Interventions

| Group/Arm Name | Group/Arm A | Group/Arm B | Group/Arm C |
|---|---|---|---|
| Intervention Name | Guselkumab | Apremilast | Placebo |
| | Liquid provided in a | Encapsulated pills | Liquid provided in |
| | single-use prefilled | | single use PFS |
| | syringe (PFS) | | AND Encapsulated |
| | assembled with the | | pills |
| | UltraSafe PLUSTM | | |
| | Passive Needle Guard | | |
| | (PFS-U) | | |
| Unit Dose | 100 mg | 10 mg, 20 mg, | N/A |
| Strength(s) | | and 30 mg | |
| Dosage Level(s) | 100 mg at Week 0, | Day 1: 10 mg in | |
| and Frequency | 4 and then q8w | morning | |
| | | Day 2: 10 mg in | |
| | | morning and | |
| | | 10 mg in evening | |
| | | Day 3: 10 mg in | |
| | | morning and | |
| | | 20 mg in evening | |
| | | Day 4: 20 mg in | |
| | | morning and | |
| | | 20 mg in evening | |
| | | Day 5: 20 mg in | |
| | | morning and | |
| | | 30 mg in evening | |
| | | Day 6 and thereafter: | |
| | | 30 mg twice daily | |
| Route of | Subcutaneous | Oral | Oral |
| Administration | | | Subcutaneous |
| Dosing instructions | Group A: | Group B: | Group C: |
| | Subcutaneous | Study agent will be | Subcutaneous |
| | study agent will be | dispensed in | placebo agent will be |
| | administered by site | treatment blister | administered by site |
| | personnel at Weeks 0 | cards which each | personnel. Oral |
| | and 4. Beginning at | contain 4 weeks of | placebo agent will be |
| | Week 28, at the | treatment. The initial | dispensed in |
| | discretion of the | blister card dispensed | treatment blister |
| | investigator and | at Week 0 will | cards which each |
| | participant, and after | contain 10, 20, and | contain 4 weeks of |
| | appropriate and | 30 mg apremilast | treatment. |
| | documented training, | tablets per standard | |
| | participants may | dose titration as listed | |
| | selfadminister study | in the protocol | |
| | agent at the | followed by 30 mg | |
| | investigative site | BID. All remaining | |
| | under the supervision | treatment blister | |
| | of an HCP. A | cards will contain 30 | |
| | caregiver may also be | mg BID. | |
| | trained to administer | | |
| | study agent. After | | |
| | Week 24, patients | | |
| | may selfadminister | | |
| | drug at home (or by a | | |
| | caregiver) if desired. | | |

### Statistical Considerations

Sample Size: A sample size of approximately 450 participants (150 participants per treatment group) will allow at least 90% power to detect treatment effect differences of 45% between guselkumab (60%) and placebo (15%) for the primary endpoint, and treatment effect differences of 30% between guselkumab (60%) and apremilast (30%) for the first major secondary endpoint assuming a 2-sided alpha level of 0.05 or 0.001.

Table 3 shows the power calculation to detect the treatment differences with some assumptions for the primary endpoint and first major secondary endpoint. For the other multiplicity controlled major secondary endpoints, based on the available data from the studies referenced below (Table 4), assuming a treatment effect difference of about 35% to 70%, a sample size of 150 per arm will allow at least 90% power.

TABLE 3
Power calculation for primary endpoint and first ranked major secondary endpoint

| | | Placebo | Guselkumab | | |
|---|---|---|---|---|---|
| | Alpha | (N = 150) | (N = 150) | Delta | Power |
| Primary endpoint | | | | | |
| Proportion of participants who achieve an | 0.05 | 15% | 50% | 35% | >99% |
| IGA score of 0 or 1 with at least ≥2 grade | | | 60% | 45% | >99% |
| improvement from baseline at Week 16 | 0.001 | 15% | 50% | 35% | >99% |
| | | | 60% | 45% | >99% |
| Major secondary endpoint | | | | | |
| Proportion of participants who achieve an | 0.05 | 30% | 55% | 25% | 99% |
| IGA score of 0 or 1 with at least ≥2 grade | | | 60% | 30% | >99% |
| improvement from baseline at Week 16 | 0.001 | 30% | 55% | 25% | |
| | | | 60% | 30% | |

TABLE 4
Reference studies for primary endpoint and selected major secondary endpoints

| | | Guselkumab | Guselkumab |
|---|---|---|---|
| Endpoints: All at Week 16 | Reference | vs Placebo | vs Apremilast |
| Proportion of participants who achieve an IGA score | VOYAGE | 82.9% vs 9.1% | |
| of 0 or 1 with ≥2 grade improvement from baseline | 1 & 2 | | |
| (BSA 10-15%) | | | |
| Proportion of participants who achieve an IGA score | DISCOVER | 55.7% vs 12.2% | |
| of 0 or 1 with ≥2 grade improvement from baseline | 1 & 2 | | |
| (BSA 3-15%) | | | |
| Proportion of participants who achieve an Static | DISCOVER | | 55.7% vs 21.6% |
| Physician Global Assessment (sPGA) score of 0 or 1 | 1 & 2 vs | | |
| with at least ≥2 grade improvement from baseline at | ADVANCE | | |
| Week 16 (BSA 2-15%) | | | |
| Proportion of participants who achieve an IGA score | VOYAGE 1 | 47.7% vs 1.1% | |
| of clear (0) at Week 16 (BSA >=10%) | | | |
| Proportion of participants who achieve a PASI 90 | VOYAGE 1 | 73.3% vs 2.9% | |
| response | | | |
| Proportion of participants who achieve a PASI 90 | VOYAGE 2 | 70.0% vs 2.4% | |
| response | | | |
| Proportion of participants who achieve a PASI 90 | VOYAGE 1 vs | | 73.3% vs 9.8% |
| response | ESTEEM1 | | |
| Proportion of participants who achieve a PASI 90 | VOYAGE 1 vs | | 73.3% vs 8.8% |
| response | ESTEEM2 | | |
| Proportion of participants who achieve a PASI 100 | VOYAGE 1 | 37.4% vs 0.6% | |
| response | | | |
| Proportion of participants who achieve a ss-IGA | VOYAGE 1 | 83.4% vs 14.5% | |
| score of 0 or 1 with ≥2-grade improvement from | | | |
| baseline among randomized participants with scalp | | | |
| psoriasis and an ss-IGA score ≥2 at baseline | | | |
| Proportion of participants who achieve a ss-IGA | VOYAGE 1 vs | | 83.4% vs 46.5% |
| score of 0 or 1 with ≥2-grade improvement from | ESTEEM1 | | |
| baseline among randomized participants with scalp | | | |
| psoriasis and an ss-IGA score ≥2 at baseline | | | |
| Proportion of participants with ≥4-point reduction | VOYAGE 1 | 75.0% vs 5.7% | |
| (improvement) in PSSD Itch score from baseline | | | |
| among participants with a PSSD Itch score ≥4 at | | | |
| baseline | | | |
| Change in PSSD Symptom score from baseline: | VOYAGE 1 | −41.9 (24.61) vs | |
| Mean (SD) | | −3.0 (19.56) | |
| Percent improvement in NAPSI score from | VOYAGE 1 | 34.37 (42.448) vs | |
| baseline: Mean (SD) | | −0.93 (57.893) | |

### Statistical Methods:

Descriptive statistics (e.g., mean, median, standard deviation [SD], interquartile [IQ] range, minimum, and maximum) will be used to summarize continuous variables. Counts and percentages will be used to summarize categorical variables. Graphical data displays (eg, line plots) may also be used to summarize data.

A Cochran-Mantel-Haenszel (CMH) chi-squared statistical test will be used for the primary endpoint. In the primary efficacy analysis, data from all randomized participants will be analyzed according to their assigned group. Participants who discontinue study intervention due to lack of efficacy or due to an AE of worsening of psoriasis, initiate prohibited medication or therapy that could improve psoriasis before Week 16 will be considered nonresponders for the primary endpoint at Week 16.

Analyses suitable for categorical data (e.g., chi-square tests, CMH chi-square tests, or logistic regression, as appropriate) will be used to compare the proportions of participants achieving selected endpoints (e.g., clinical response). In cases of rare events, the Fisher's exact test will be used for treatment comparisons. Continuous response parameters will be compared using an analysis of variance (ANOVA) or analysis of covariance (ANCOVA), unless otherwise specified. If the normality assumption is in question, an ANOVA or ANCOVA on the van der Waerden normal scores will be used.

The overall Type I error rate will be controlled at the significance level of 0.05 (2-sided).

The Invention can be Described with Reference to the Following Numbered Embodiments:
1. The use of an antibody specific to IL23 for treatment of mild to moderate psoriasis in a patient, wherein the antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3, and the use results in a clinical response in the patient.
2. The use of embodiment 1, wherein the antibody is administered in an initial dose, a dose about 4 weeks after the initial dose and a dose about 12 weeks after the initial dose.
3. The use of embodiment 2, wherein the antibody is administered subcutaneously.
4. The use of embodiment 1, wherein the initial dose and the doses about 4 weeks after the initial dose and about 12 weeks after the initial dose are 100 mg of the antibody.
5. The use of embodiment 1, wherein the patient is a responder to the antibody and is identied as meeting a clinical and/or exploratory endpoint, wherein the clinical endpoint is from the group consisting of:
(i) achievement of an IGA score of cleared (0) or minimal (1) with at least ≥2 grade improvement from baseline;
(ii) achievement of BSA≤1%;
(iii) achievement of an IGA score of cleared (0);
(iv) achievement of a PASI 90 response;
(v) achievement of a PASI 100 response;
(vi) achievement of a Scalp-Specific Investigator's Global Assessment (ss-IGA) score of absence of disease (0) or very mild disease and have at least a 2-grade improvement from baseline and an ss-IGA score ≥2 at baseline;
(vii) achievement of ≥4-point reduction (improvement) in Psoriasis Symptom and Sign Diary (PSSD) Itch score from baseline among participants with a PSSD Itch score ≥4 at baseline;
(viii) achievement of a PSSD symptom score of 0, among randomized participants with a baseline PSSD symptom score ≥1; and
(ix) percent improvement from baseline in the Nail Psoriasis Severity Index (NAPSI) among randomized participants with nail psoriasis at baseline.
6. The use of embodiment 5, wherein the clinical endpoint(s) is measured about 16, 24, 48 and/or 96 weeks after initial treatment.
7. The use of embodiment 6, wherein the clinical endpoint(s) is measured about 16 weeks after initial treatment.
8. The use of any of embodiments 5-7, wherein the clinical endpoints are compared to clinical endpoints of patients being treated with apremilast.
9. The use of embodiment 1, wherein the antibody comprises a light chain variable region amino acid sequence of SEQ ID NO: 8 and a heavy chain variable region amino acid sequence of SEQ ID NO: 7.
10. The use of embodiment 1, wherein the antibody comprises a light chain amino acid sequence of SEQ ID NO: 10 and a heavy chain amino acid sequence of SEQ ID NO: 9.
11. The use of embodiment 9 or 10, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.
12. The use of embodiment 1, further comprising use of one or more additional drugs used to treat mild to moderate psoriasis.
13. The use of embodiment 12, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.
14. Use of an antibody specific to IL23 for the treatment of mild to moderate psoriasis in a patient, (i) in an initial 100 mg subcutaneous dose of the antibody, (ii) a 100 mg subcutaneous dose of the antibody about 4 weeks after the initial dose, and (iii) a 100 mg subcutaneous dose of the antibody about every 8 weeks after the dose at about 4 weeks after the initial dose, wherein the antibody comprises a light chain variable region amino acid sequence of SEQ ID NO: 8 and a heavy chain variable region amino acid sequence of SEQ ID NO: 7 and the patient is a responder to the antibody by being identied as meeting a clinical endpoint about 16 weeks after the initial dose, wherein the clinical endpoint is selected from the group consisting of: (i) achievement of an IGA score of cleared (0) or minimal (1) with at least ≥2 grade improvement from baseline; (ii) achievement of BSA≤1%; (iii) achievement of an IGA score of cleared (0); (iv) achievement of a PASI 90 response and (v) achievement of a PASI 100 response.

What is claimed is:

1. A method of treating mild to moderate psoriasis in a patient, comprising administering to the patient an antibody specific to IL23, wherein the antibody comprises a light chain variable region and a heavy chain variable region, said light chain variable region comprising:
a complementarity determining region light chain 1 (CDRL1) amino acid sequence of SEQ ID NO:4;
a CDRL2 amino acid sequence of SEQ ID NO:5; and
a CDRL3 amino acid sequence of SEQ ID NO:6,
said heavy chain variable region comprising:
a complementarity determining region heavy chain 1 (CDRH1) amino acid sequence of SEQ ID NO:1;
a CDRH2 amino acid sequence of SEQ ID NO:2; and
a CDRH3 amino acid sequence of SEQ ID NO:3, wherein the patient is a responder to the antibody.

2. The method of claim 1, wherein the antibody is administered in an initial dose, a dose about 4 weeks after the initial dose and a dose about 12 weeks after the initial dose.

3. The method of claim 2, wherein the initial dose and the doses about 4 weeks after the initial dose and about 12 weeks after the initial dose are about 100 mg of the antibody.

4. The method of claim 3, wherein the antibody is administered subcutaneously.

5. The method of claim 4, further comprising administering a maintenance dose of the antibody about every 8 weeks after administration of the dose about 12 weeks after the initial dose.

6. The method of claim 1, wherein the patient is a responder to the antibody by being identied as meeting a clinical endpoint.

7. The method of claim 6, wherein the clinical endpoint is selected from the group consisting of:
achievement of an IGA score of cleared (0) or minimal (1) with at least ≥2 grade improvement from baseline;
(ii) achievement of BSA≤1%;
(iii) achievement of an IGA score of cleared (0);
(iv) achievement of a PASI 90 response;
(v) achievement of a PASI 100 response;
(vi) achievement of a Scalp-Specific Investigator's Global Assessment (ss-IGA) score of absence of disease (0) or very mild disease and have at least a 2-grade improvement from baseline and an ss-IGA score ≥2 at baseline;
(vii) achievement of ≥4-point reduction (improvement) in Psoriasis Symptom and Sign Diary (PSSD) Itch score from baseline among participants with a PSSD Itch score ≥4 at baseline;
(viii) achievement of a PSSD symptom score of 0, among randomized participants with a baseline PSSD symptom score ≥1; and
(ix) percent improvement from baseline in the Nail Psoriasis Severity Index (NAPSI) among randomized participants with nail psoriasis at baseline.

8. The method of claim 7, wherein the clinical endpoint is measured about 16 weeks after the initial dose.

9. The method of claim 7, wherein the clinical endpoint(s) is measured about 24 weeks, 52 weeks and/or 104 weeks after initial treatment.

10. The method of claim 9, wherein the clinical endpoint(s) is measured about 24 weeks after initial treatment.

11. The method of any of claims 7-10, wherein the clinical endpoints are compared to clinical endpoints of patients being treated with apremilast.

12. The method of claim 1, wherein the antibody comprises a light chain variable region amino acid sequence of SEQ ID NO: 8 and a heavy chain variable region amino acid sequence of SEQ ID NO: 7.

13. The method of claim 1, wherein the antibody comprises a light chain amino acid sequence of SEQ ID NO: 10 and a heavy chain amino acid sequence of SEQ ID NO: 9.

14. The method of claim 12 or 13, wherein the antibody is in a composition comprising 7.9% (w/v) sucrose, 4.0 mM Histidine, 6.9 mM L-Histidine monohydrochloride monohydrate; 0.053% (w/v) Polysorbate 80 of the pharmaceutical composition; wherein the diluent is water at standard state.

15. The method of claim 1, further comprising administering to the patient one or more additional drugs used to treat mild to moderate psoriasis.

16. The method of claim 15, wherein the additional drug is selected from the group consisting of: immunosuppressive agents, non-steroidal anti-inflammatory drugs (NSAIDs), methotrexate (MTX), anti-B-cell surface marker antibodies, anti-CD20 antibodies, rituximab, TNF-inhibitors, corticosteroids, and co-stimulatory modifiers.

17. A method of treating mild to moderate psoriasis in a patient, comprising administering to the patient (i) an initial subcutaneous dose of 100 mg of an antibody specific to IL23, (ii) a 100 mg subcutaneous dose of the antibody about 4 weeks after the initial dose, (iii) a 100 mg subcutaneous dose of the antibody about 12 weeks after the initial dose, and (iv) a 100 mg subcutaneous dose of the antibody about every 8 weeks after the dose at about 12 weeks after the initial dose, wherein the antibody comprises a light chain variable region amino acid sequence of SEQ ID NO: 8 and a heavy chain variable region amino acid sequence of SEQ ID NO: 7 and the patient is a responder to the antibody by being identied as meeting a clinical endpoint about 24 weeks after the initial dose, wherein the clinical endpoint is change from baseline in Modified Rodnan Skin Score (mRSS).
